(19) United States
(12) Patent Application Publication
(10) Pub. No.: US 2026/0216236 A1
(43) Pub. Date: Jul. 30, 2026

(54) COMPOSITIONS AND METHODS FOR TREATING HYPERPROCALCITONEMIA

(71) Applicant: Vivacelle Bio, Inc., Kansas City, MO (US)

(72) Inventor: Cuthbert O. SIMPKINS, Kansas City, MO (US)

(21) Appl. No.: 19/146,687

(22) PCT Filed: Jan. 18, 2024
(86) PCT No.: PCT/US2024/011961

§ 371 Date: Jul. 9, 2025

Related U.S. Application Data
(60) Provisional application No. 63/482,540, filed on Jan. 31, 2023.

(51) Int. Cl. A61K 33/14; A61K 9/107; A61K 9/127; A61K 31/4172; A61K 33/00; A61K 38/38; A61K 47/10; A61K 47/22; A61K 47/24; A61K 47/42; A61K 47/44; A61P 5/14

(52) U.S. Cl. CPC A61K 33/14; A61K 9/1075; A61K 9/127; A61K 31/4172; A61K 33/00; A61K 38/38; A61K 47/10; A61K 47/22; A61K 47/24; A61K 47/42; A61K 47/44; A61P 5/14

(57) ABSTRACT

A composition for treating hyperprocalcitonemia is described. The composition comprises a lipophilic or hydrophobic component, an amphiphilic emulsifier, a polar liquid carrier, and with or without one or more electrolytes, where the amphiphilic emulsifier forms micelles having a lipophilic or hydrophobic core comprising the lipophilic or hydrophobic component in the polar liquid carrier, and/or liposomes organized as a lipid bilayer and/or other particle configurations.

### RELATED APPLICATIONS

This application is a National Stage application which claims priority from the International Application PCT/US24/11961, filed Jan. 18, 2024, which claims priority of U.S. Provisional Patent Application No. 63/482,540, filed on Jan. 31, 2023. The entirety of the aforementioned applications are incorporated herein by reference.

### FIELD

The present application relates generally to pharmaceutical compositions and, in particular, to compositions for treating hyperprocalcitonemia.

### BACKGROUND

Procalcitonin is a 116 amino acid peptide with an approximate molecular weight of 13.0 kDa. Procalcitonin is expressed in the parafollicular cells of the thyroid gland where it is enzymatically cleaved to produce the calcium regulating hormone, calcitonin. Normally the production of procalcitonin is limited to these cells and its concentration in the bloodstream is very low at <0.05 ng/ml. Under conditions of severe physiological stress, monocytes and parenchymal cells of many organs such as fat, liver, lung, muscle, stomach, kidney, and brain can synthesize procalcitonin resulting in a 100,000-fold increase in blood concentration. The enzyme that transforms procalcitonin to calcitonin is absent from tissues other than the thyroid. This results in the release of procalcitonin instead of calcitonin into the bloodstream in times of physiological stress. Recent studies have demonstrated that procalcitonin is more than a marker of the inflammatory response. It is also a mediator of this response, thus making it a target for the treatment of diseases in which its concentration is increased.

The inflammatory response is the pathophysiological basis of a broad range of medical conditions. These medical conditions may or may not involve infection. Examples are bacterial or viral infections, malaria, blood loss, severe heart failure, injury to the brain and/or spinal cord, bone fractures, non-orthopedic trauma, pancreatitis, burns, ischemic stroke, subarachnoid hemorrhage, heat stroke, vasculitis, metastatic cancer, dementia, sarcopenia, aging, chronic or acute kidney disease, chronic or acute liver failure, heart failure, acute respiratory distress syndrome and arthritis. In vitro and in vivo studies have revealed the role of procalcitonin as a mediator of inflammation and tissue injury that is characteristic of these numerous medical conditions.

Procalcitonin has been shown to increase the intracellular concentration of calcium, which is similar to the proinflammatory cytokine interleukin 8 (Becker et al. Procalcitonin in sepsis and systemic inflammation: a harmful biomarker and a therapeutic target. Br. J. Pharmacol. 2010 159 (2): 253-264).

Studies with rat aortic vascular smooth muscle cells showed that procalcitonin did not directly affect nitric oxide release from these cells. However, when combined with lipopolysaccharide, tumor necrosis factor and interferon γ, procalcitonin greatly increased the production of nitric oxide from these cells (Tilg, Peschel Interferon-alpha and its effects on the cytokine cascade: a pro and anti-inflammatory cytokine. Leuk. Lymphoma 1996 23(1-2): 55-60). Therefore, procalcitonin is not a primary inducer, but it is an amplifier of the inflammatory response. In addition, procalcitonin has direct effects that make it a toxic mediator.

Examples of procalcitonin as a toxic mediator are replete in the literature. Wagner et al. (Procalcitonin Impairs Endothelial Cell Function and Viablity. 2017; 124 (3): 835-845) demonstrated that procalcitonin induces endothelial barrier disruption, cell migration, new capillary formation and endothelial cell death. In addition, in mice recovery of hindlimb perfusion in an ischemic leg was impaired. Sauer et al. (Procalcitonin Impairs Liver Cell Viability and Function In Vitro: A Potentially New Mechanism of Liver Dysfunction and Failure during Sepsis? Biomed Research International Volume 2017: Article 6130725 Open Access) found that procalcitonin inhibited hepatocyte proliferation and induced hepatocyte death. The role of procalcitonin as a toxic mediator was further demonstrated using procalcitonin deficient mice. These mice were protected from septic shock and showed decreased pulmonary inflammation (Baranowsky et al. Procalcitonin Exerts a Mediator Role in Septic Shock Through the Calcitonin Gene-Related Peptide Receptor. Crit. Care Med. 2021 49(e41-e52)). Sullivan and Schmidt elucidated the impairment of the microcirculation by procalcitonin (Procalcitonin: A Mediator of Microvascular Dysfunction during Critical Illness. American Journal of Respiratory and Critical Care Medicine 2022 206(4):375-376). Infusion of procalcitonin into septic animals increased mortality and administration of procalcitonin antibodies decreased mortality of septic animals (Becker et al. Procalcitonin in sepsis and systemic inflammation: a harmful biomarker and a therapeutic target. Br. J. Pharmacol. 2010 159 (2): 253-264).

In humans, the lower blood procalcitonin has been shown to indicate a favorable prognosis in numerous conditions, such as sepsis, acute respiratory distress syndrome, fractures, acute kidney injury, heart failure and trauma. Given its action as an amplifier of the inflammatory response and the direct adverse effects of procalcitonin, agents that reduce procalcitonin would be beneficial in acute or chronic conditions in which procalcitonin is elevated.

### SUMMARY

One aspect of the application is a method for treating hyperprocalcitonemia in a subject in need thereof. The method comprises the step of administering to a subject in need of the treatment, an effective amount of a nanoparticle composition comprising: an amphiphilic emulsifier in an amount of 0.1%-100% (w/v); a lipophilic or hydrophobic component in an amount of 0-35% (w/v); a polar liquid carrier; and one or more electrolytes, wherein the nanoparticle composition comprises liposomes and/or micelles having a mean diameter of 1-800 nm.

Another aspect of the present application relates to a method for treating a disease or condition associated with elevated procalcitonin in a subject including but not limited to chronic or acute kidney failure, chronic or acute liver failure, chronic or acute respiratory failure, sarcopenia of aging, dementia, orthopedic or non-orthopedic trauma, surgical procedures, myocardial infarction, autism, ischemic stroke, Parkinson's disease, vasculitis, bone fractures, blood loss and depression. The method comprises the step of administering to a subject in need of treatment of the hyperprocalcitonemia, an effective amount of a hydrophobic nanoparticle composition comprising: an amphiphilic emulsifier in an amount of 0.1%-100% (w/v); a lipophilic or hydrophobic component in an amount of 0-35% (w/v); a polar liquid carrier; and one or more electrolytes, wherein the nanoparticle composition comprises liposomes and/or micelles having diameters in the range of 1-800 nm.

Another aspect of the present application relates to a method for treating aging. The method comprises the step of administering to a subject in need of treatment, an effective amount of nanoparticle composition comprising: an amphiphilic emulsifier in an amount of 0.1%-100% (w/v); a lipophilic or hydrophobic component in an amount of 0-35% (w/v); a polar liquid carrier; and one or more electrolytes, wherein the nanoparticle composition comprises liposomes and/or micelles having diameters in the range of 1-800 nm.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

### I. Definitions

The term “acute critical illness”, is meant to include any condition rendering the patient in urgent need for intensive care therapy. The condition may be caused by any acute and extensive injurious hit to the body including but not limited to physical trauma, burn injury trauma, infection (hereunder sepsis, severe sepsis, blood loss), systemic inflammatory response syndrome (SIRS), acute myocardial infarction, diabetic ketoacidosis or other thromboembolic events.

The term “chronic illness”, is meant to include any condition that does not need urgent therapy and that is persistent or long-lasting in its effects or that comes with time. The condition may be caused by any long-lasting disease including but not limited to arthritis, chronic renal failure, liver failure, cancer metastases, dementia, Parkinson's disease or schizophrenia.

The term “intensive care therapy”, also term “organ supportive care” here, may include but is not limited to ventilation therapy, (e.g., mechanical ventilation), hemodialysis, vasopressor therapy, fluid therapy, blood transfusion therapy with administration of red blood cell concentrates, fresh frozen plasma, platelet concentrates, whole blood or coagulation factor concentrates, systemic antibiotic and/or antiviral and/or antifungal and/or antiprotozoic therapy, parenteral nutrition, granulocyte infusion, T cell infusion, stem cell infusion, anticoagulant and/or antithrombotic therapy including low molecular weight heparins, administration of corticosteroids, tight glycemic control, as well as the treatment with ventricular assist devices, intra-aortic balloon pumps, plasmapheresis, extracorporeal membrane oxygenation, and rotary mechanical circulatory support systems, etc.

The term “trauma” as used herein means any shock or body wound produced by a physical injury such as accident, injury or impact to living tissue caused by an extrinsic agent such as blast trauma, crush injury, blunt trauma, penetrating trauma, trauma caused by chemical injury (spills, warfare or intoxication), radiation or burns.

As used herein, the term “hyperprocalcitonemia” refers to a condition in which a person has higher than normal levels of the hormone prolactin in the blood. In some embodiments, hyperprocalcitonemia refers to a condition in which a person has a blood procalcitonin level of 0.05 ng/ml or higher.

As used herein, the terms “treatment” and “treating” refer to the management and care of a patient with hyperprocalcitonemia or at risk for developing hyperprocalcitonemia. The term is intended to include the full spectrum of treatments for this condition, such as administration of the phospholipid nanoparticle compositions of the present application for the purpose of: ameliorating, alleviating or relieving symptoms or complications; delaying the progression of the condition, disease or disorder; curing or eliminating the condition, disease or disorder; and/or reducing the risk of or preventing the condition, disease or disorder, including preventing recurrence of the disease, wherein “preventing” or “prevention” is to be understood to refer to the management and care of a patient for the purpose of hindering the development of the condition, disease or disorder, and includes the administration of the nanoparticle compositions to prevent the onset of symptoms or complications. The individual to be treated is a human being or non-human animal. An individual to be treated according to the present application can be of various ages and/or sexes.

The term “organ failure” refers to an altered organ function in an acutely ill patient requiring medical intervention to achieve body homeostasis and/or to compensate for the loss of function from that failing organ. The organs include but are not limited to heart and vessels (cardiac failure, vasoplegia), lungs (respiratory failure), liver (liver failure), kidneys (renal failure), brain (encephalopathy), bones (arthritis) or brain (dementia, encephalitis or meningitis).

The term “sepsis” is used in the conventional clinical meaning, referring to a whole-body inflammatory state (called systemic inflammatory response syndrome (SIRS)) AND the presence of a known or suspected infection. “Severe sepsis” is defined as sepsis-induced organ dysfunction or tissue hypoperfusion (manifesting e.g., as elevated procalcitonin, tumor necrosis factor or interleukin-1, vasoplegia, elevated lactate, decreased urine output or altered mental status). Sepsis can lead to severe sepsis, multiple organ dysfunction syndrome/multiple organ failure (MODS) and death.

The term “systemic inflammatory response syndrome” or “SIRS” is used in the conventional clinical meaning, referring to systemic inflammation in response to an insult without confirmed infectious process. SIRS can be diagnosed when 2 or more of the following criteria are present: 1) Body temperature less than 36° C. (96.8° F.) or greater than 38° C. (100.4° F.); 2) Heart rate greater than 90 beats per minute; 3) Tachypnea (high respiratory rate), with greater than 20 breaths per minute or an arterial partial pressure of carbon dioxide less than 4.3 kPa (32 mmHg); and 4) White blood cell count less than 4000 cells/mm³ (4×10⁹ cells/L) or greater than 12,000 cells/mm³ (12×10⁹ cells/L) or the presence of greater than 10% immature neutrophils (band forms). When an infection is suspected or proven (by culture, stain, or polymerase chain reaction (PCR)), together with SIRS, this is per definition sepsis. Hyperprocaclcitonemia is seen with sepsis (Schuetz et al. Serial Procalcitonin Predicts Mortality in Severe Sepsis Patients: Results From the Multicenter Procalcitonin MOnitoring SEpsis (MOSES) Study. Crit Care Med. 2017 20(30): Open Access), severe blood loss (Procalcitonin release patterns in a baboon model of trauma and sepsis: relationship to cytokines and neopterin. Crit. Care Med. 2000. 28(11):3659-3663), severe heart failure (Picariello et al. Procalcitonin in patients with acute coronary syndromes and cardiogenic shock submitted to percutaneous coronary intervention. Intern Emerg Med. 2009 4(5):403-408). After injury to the brain and spinal cord interleukin 6 and during status epilepticus interleukin 6 and procalcitonin have been shown to be increased (Abdulla et al. Neurogenically Originated Inflammatory Response Syndrome: Role in the Neurocritical Patient. J.Neurointensive Care 2022 5(2): 39-43). Therefore, physiological disruption induced by infection, blood loss, heart failure or neurological injury are different with respect to their initiation. However, after initiation they all have a common inflammatory component that leads to the production of procalcitonin.

The term “systemic inflammation” is altered organ function in an acutely or chronically ill patient due to the nonspecific conserved response of the body (vasculature, immune system, tissues) to infections, non-infectious antigens, trauma, burn, organ/tissue destruction/degeneration/damage, ischemia, haemorrhage, intoxication, and/or malignancy.

The terms “micelle” and “lipid carrying micelle (LM)” are used interchangeably herein with reference to an aggregate of molecules dispersed in a liquid, including an aggregate with the hydrophilic “head” regions in contact with surrounding solvent, sequestering the hydrophobic single tail regions in the micelle center, which forms a hydrophobic core suitable for containing and delivering hydrophobic agents.

The term “liposome” as used herein refers to a vesicular structure comprised of a lipids having a tail group comprising a long hydrophobic hydrocarbon chain and a hydrophilic head group. The lipids are arranged to form a lipid bilayer with an inner aqueous core environment suitable for containing and delivering aqueous agents and a lipid wall suitable for containing hydrophobic agents, especially gasses such as oxygen.

The term hydrophobic particle includes micelles and liposomes but also includes any particle that creates a hydrophobic space or contains a hydrophobic substance such as a hydrogel or a polycyclic compound with a cavity for encapsulating a hydrophobic species ie a molecular cage.

### II. Methods of Treatment

One aspect of the present application relates to a method for treating or preventing hyperprocalcitonemia in a patient, comprising administering to the subject an effective amount of the nanoparticle composition of the present application. The inventor of the present application has unexpectedly found that administration of the nanoparticle composition of the present application can prevent or reduce hyperprocalcitonemia.

Based on the fund of knowledge in the field, infusion of a hydrophobic nanoparticle composition should increase procalcitonin. Instead, we have found a large and consistent reduction. Procalcitonin is a 116 amino acid peptide with an approximate molecular weight of 13.0 kDa. Procalcitonin is expressed in the parafollicular cells of the thyroid gland where it is enzymatically cleaved to produce the calcium regulating hormone, calcitonin. Normally the production of procalcitonin is limited and its concentration in the bloodstream is very low at <0.05 ng/ml. The enzyme that transforms procalcitonin to calcitonin is absent from tissues other than the thyroid. However, under conditions of severe physiological stress monocytes and parenchymal cells of many organs can synthesize procalcitonin resulting in a 100,000-fold increase in blood concentration. Procalcitonin elevation is seen in a wide array of conditions such as sepsis, acute renal failure, chronic renal failure, Alzheimer's disease, vascular dementia, dementia with Lewy bodies, frontotemporal dementia, encephalitis, meningitis, acute respiratory distress syndrome, COVID-19 infections, pre-eclampsia, lung cancer, colorectal cancer, ovarian cancer, cardiac failure, severe blood loss and trauma. Procalcitonin has been viewed as a marker of the severity of disease. But it has also been shown that procalcitonin itself has injurious effects. Procalcitonin elevation occurs as a result of the increase in proinflammatory proteins such as tumor necrosis factor and interleukin 6 (Whang et al. Procalcitonin and proinflammatory cytokine Interactions in Sepsis. Shock 2000. 14(1):73-78 and Nijsten et al. Procalcitonin behaves as a fast responding acute phase protein in vivo and in vitro. Crit. Care Med. 2000 28(2):458-461). Cai et al. (Mediators of Inflammation 2010 Volume 2010 Article ID642462 (open access)) showed that both sepsis and severe blood loss induce tumor necrosis factor and high-mobility box 1 protein. Furthermore, Reidl et al. (Crit. Care Med. 2000. 28(11):3659-3663) showed that procalcitonin is increased in both sepsis and severe blood loss.

Also, Hierholzer et al. (Am. J. Physiol. 1998 275(3):L611-621) showed that severe blood loss led to an increase in interleukin 6. The infusion of hydrophobic nanoparticles comprised of liposomes and micelles such as Intralipid has been shown to increase the blood concentration of proinflammatory mediators. Krough-Madesen et al. (Am J. Physiol Endocrinol Metab 2008, 294(2) E371-379) showed that Intralipid increased the production of proinflammatory mediators such as tumor necrosis factor and interleukin 6 after endotoxin infusion. Lou et al. (Mol Nutr Food Res. 2021 65(5)), found that Intralipid infusion increased numerous proinflammatory mediators such as interleukin 6. Infusion of Intralipid into pregnant rats increased proinflammatory mediators as shown by Duncan et al. (FASEB Journal 2019 33 (51)). The increase in proinflammatory mediators by Intralipid would bring one skilled in the art to the expectation that the infusion of the nanoparticle composition of the present application would lead to an increase in procalcitonin. In addition, because nanoparticle composition increases proinflammatory mediators, one skilled in the art would not give a patient nanoparticle composition of the present application with the expectation that procalcitonin with its harmful effects would be reduced. Yet contrary to the expectation created by the literature, we have found that procalcitonin consistently decreases after the infusion of the nanoparticle composition of the present application. It is also remarkable that no serious adverse effects attributable to nanoparticle composition of the present application have been observed. Therefore, the unexpected reduction of procalcitonin which itself promotes organ injury by nanoparticle composition of the present application has significant therapeutic applicability.

The nanoparticle compositions of the present application may be used to treat or prevent hyperprocalcitonemia caused by a plurality of different disease conditions affected by tissue injury, including but not limited to sepsis, major trauma, burns, pancreatitis, aspiration syndromes, extracorporeal circulation (e.g., cardiac bypass), multiple blood transfusion, ischemia-reperfusion injury, autoimmune disease, severe blood loss, heat-induced illness, eclampsia, poisoning/toxicity. In some embodiments, the nanoparticle compositions of the present application are used to treat or prevent hyperprocalcitonemia resulting from sepsis caused by flu virus or coronavirus (such as SARS, MERS and COVID 19 viruses) infection.

A chronic pro-inflammatory status is a pervasive feature of aging. This chronic low-grade inflammation occurring in the absence of overt infection has been defined as “inflammagin” and represents a significant risk factor for morbidity and mortality in the elderly. The nanoparticle compositions of the present application may be used to treat aging. In some embodiments, the method comprises the step of administering to a subject in need of treatment, an effective amount of nanoparticle composition comprising: an amphiphilic emulsifier in an amount of 0.1%-100% (w/v); a lipophilic or hydrophobic component in an amount of 0-35% (w/v); a polar liquid carrier; and one or more electrolytes, wherein the nanoparticle composition comprises liposomes and/or micelles having diameters in the range of 1-800 nm.

The nanoparticle composition of the present application may be administered intravenously, intra-arterially, intraosseously or intracardially to a subject in need of such treatment. In certain embodiments, the nanoparticle composition is administered in an amount of 50-5000 ml, 50-4000 ml, 50-3000 ml, 50-2000 ml, 50-1000 ml, 50-500 ml, 100-5000 ml, 100-4000 ml, 100-3000 ml, 100-2000 ml, 100-1000 ml, 100-500 ml, 200-5000 ml, 200-4000 ml, 200-3000 ml, 200-2000 ml, 200-1000 ml, 200-500 ml, 500-5000 ml, 500-4000 ml, 500-3000 ml, 500-2000 ml, 500-1000 ml, 1000-5000 ml, 1000-4000 ml, 1000-3000 ml and 1000-2000 ml. In some embodiments, the nanoparticle composition is administered in an amount equal to about 10%, 15%, 20%, 25%, 30%, 35%, 40%, 45%, 50%, 55% 60%, 65%, 70%, 75%, 80%, 85% or 90% of the normal blood volume of a subject in a period of 30 seconds to 24 hours. In some embodiments, the nanoparticle composition of the present invention is administered at multiple doses with an interval of 2, 4, 8, 12, 24 or 48 hours between doses.

In some embodiments, the nanoparticle composition is given at a rate of 0.1-5000 ml/min, 0.1-2000 ml/min, 0.1-1000 ml/min, 0.1-500 ml/min, 0.1-200 ml/min, 0.1-100 ml/min. 0.1-50 ml/min, 0.1-20 ml/min, 0.1-10 ml/min, 0.1-5 ml/min, 0.1-2 ml/min, 0.1-1 ml/min, 1-5000 ml/min, 1-2000 ml/min, 1-1000 ml/min, 1-500 ml/min, 1-200 ml/min, 1-100 ml/min. 1-50 ml/min, 1-20 ml/min, 1-10 ml/min, 1-5 ml/min, 1-2 ml/min, 2-5000 ml/min, 2-2000 ml/min, 2-1000 ml/min, 2-500 ml/min, 2-200 ml/min, 2-100 ml/min. 2-50 ml/min, 2-20 ml/min, 2-10 ml/min, 2-5 ml/min, 5-5000 ml/min, 5-2000 ml/min, 5-1000 ml/min, 5-500 ml/min, 5-200 ml/min, 5-100 ml/min. 5-50 ml/min, 5-20 ml/min, 5-10 ml/min, 10-5000 ml/min, 10-4000 ml/min, 10-3000 ml/min, 10-2000 ml/min, 10-1000 ml/min, 10-500 ml/min, 10-200 ml/min, 10-100 ml/min, 10-50 ml/min, 20-5000 ml/min, 20-4000 ml/min, 20-3000 ml/min, 20-2000 ml/min, 20-1000 ml/min, 20-500 ml/min, 20-200 ml/min, 20-100 ml/min, 20-50 ml/min, 50-5000 ml/min, 50-4000 ml/min, 50-3000 ml/min, 50-2000 ml/min, 50-1000 ml/min, 50-500 ml/min, 50-200 ml/min, 50-100 ml/min, 100-5000 ml/min, 100-4000 ml/min, 100-3000 ml/min, 100-2000 ml/min, 100-1000 ml/min, 100-500 ml/min, 100-200 ml/min, 200-5000 ml/min, 200-4000 ml/min, 200-3000 ml/min, 200-2000 ml/min, 200-1000 ml/min, 200-500 ml/min, 500-5000 ml/min, 500-4000 ml/min, 500-3000 ml/min, 500-2000 ml/min, 500-1000 ml/min, 1000-5000 ml/min, 1000-4000 ml/min, 1000-3000 ml/min, 1000-2000 ml/min, 2000-5000 ml/min, 2000-4000 ml/min, 2000-3000 ml/min, 3000-5000 ml/min, 3000-4000 ml/min or 4000-5000 ml/min.

In some embodiments, the nanoparticle composition is given without oxygenation. In other embodiments, the nanoparticle composition is an oxygenated nanoparticle composition. In some embodiments, the nanoparticle composition is an oxygenated nanoparticle composition with an oxygen content of 2-50, 2-40, 2-30, 2-20, 2-10, 2-5, 5-50, 5-40, 5-30, 5-20, 5-10, 10-50, 10-40, 10-30, 10-20, 15-50, 15-40, 15-30, 15-20, 20-50, 20-40, 20-30, 25-50, 25-40, 25-30, 30-50, 30-40 or 40-50 ml O₂/100 ml nanoparticle composition.

Other hemodynamic parameters, such as perfusion of brain, kidneys, heart, muscle, spleen or other tissues, cardiac output, systolic blood pressure, diastolic blood pressure, mean arterial blood pressure, stroke volume index, mitochondrial oxidative phosphorylation followed by near infrared spectroscopy or other means, blood lactate or membrane polarization, may also be used to determine the “effective amount” of the nanoparticle composition needed to treat hyperprocalcitonemia in a subject.

In some embodiments, the method further comprises administering an additional agent to the subject. The additional agent may be administered prior to, concurrently with, or after the administration of the nanoparticles of the present application. Examples of additional agents include, but are not limited to, cardioplegic and cardiotonic agents, procalcitonin antibodies, Olcegepant a calcitonin gene-related peptide antagonist, sitagliptin the dipeptidyl-peptidase 4 inhibitor to block procalcitonin signaling or activation.

### III. The Nanoparticle Composition

In one embodiment, the nanoparticle composition for treating or preventing hyperprocalcitonemia includes one or more amphiphilic emulsifiers, a lipophilic or hydrophobic component, a polar liquid carrier, and one or more electrolytes. The amphiphilic emulsifiers form lipophilic or hydrophobic substance-carrying micelles (LMs) having a lipophilic core surrounded by the polar liquid carrier, and/or liposomes containing a lipid bilayer and a hydrophilic interior (or core).

In some embodiments, the nanoparticle composition of the present application comprises LMs and liposomes having a diameter in the range of 1-1000 nm, 1-800 nm, 1-500 nm, 1-400 nm, 1-300 nm or 1-200 nm as determined by electron microscopy.

In some embodiments, the nanoparticle composition of the present application comprises (1) LMs having diameters of 30-800 nm, 30-500 nm, 30-400 nm, 30-300 nm, 30-200 nm, 30-150 nm, 30-120 nm, 30-100 nm, 30-80 nm, 40-500 nm, 40-400 nm, 40-300 nm, 40-200 nm, 40-150 nm, 40-120 nm, 40-100 nm, 40-80 nm, 50-500 nm, 50-400 nm, 50-300 nm, 50-200 nm, 50-150 nm, 50-120 nm, 50-100 nm, 50-80 nm, 100-500 nm, 100-400 nm, 10-300 nm, 100-200 nm, 100-150 nm or 100-120 nm as determined by electron microscopy, and (2) liposomes having diameters of 1-30 nm, 1-25 nm, 1-20 nm, 1-15 nm, 1-10 nm, 3-30 nm, 3-25 nm, 3-20 nm, 3-15 nm, 3-10 nm, 5-30 nm, 5-25 nm, 5-20 nm, 5-15 nm, 5-10 nm, 1-30 nm, 7-25 nm, 7-20 nm, 7-15 nm, 7-10 nm, 10-30 nm, 10-25 nm, 10-20 nm or 10-15 nm as determined by electron microscopy.

In some embodiments, the nanoparticle composition of the present application comprises nanoparticles (including both micelles and liposomes) having an average particle diameter of 1-800 nm, 1-500 nm, 1-300 nm, 1-100 nm, 1-80 nm, 1-50 nm, 1-40 nm, 1-30 nm, 1-25 nm, 1-20 nm, 1-15 nm, 1-10 nm, 1-5 nm, 5-800 nm, 5-500 nm, 5-300 nm, 5-100 nm, 5-80 nm, 5-50 nm, 5-40 nm, 5-30 nm, 5-25 nm, 5-20 nm, 5-15 nm, 5-10 nm, 10-800 nm, 10-500 nm, 10-300 nm, 10-100 nm, 10-80 nm, 10-50 nm, 10-40 nm, 10-30 nm, 10-25 nm, 10-20 nm, 15-800 nm, 15-500 nm, 15-300 nm, 15-100 nm, 15-80 nm, 15-50 nm, 15-40 nm, 15-30 nm, 15-25 nm, 15-20 nm, 20-800 nm, 20-500 nm, 20-300 nm, 20-100 nm, 20-80 nm, 20-50 nm, 20-40 nm, 20-30 nm, 20-25 nm, 25-800 nm, 25-500 nm, 25-300 nm, 25-100 nm, 25-80 nm, 25-50 nm, 25-40 nm, 25-30 nm, 30-800 nm, 30-500 nm, 30-300 nm, 30-100 nm, 30-80 nm, 30-50 nm, 30-40 nm, 40-800 nm, 40-500 nm, 40-300 nm, 40-100 nm, 40-80 nm, 40-50 nm, 50-800 nm, 50-500 nm, 50-300 nm, 50-100 nm, 50-80 nm, 80-800 nm, 80-500 nm, 80-300 nm, or 80-100 nm as determined by electron microscopy. In some embodiments, the nanoparticle composition of the present application comprises nanoparticles having an average particle diameter of 16-18 nm, 15-19 nm or 14-20 nm, as determined by electron microscopy.

In some embodiments, the nanoparticle composition of the present application comprises nanoparticles (including both micelles and liposomes) having an average diameter of 10-1000 nm, 10-800 nm, 10-500 nm, 10-300 nm, 10-200 nm, 10-150 nm, 10-120 nm, 10-100 nm, 10-90 nm, 10-70 nm, 10-50 nm, 10-30 nm, 30-1000 nm, 30-800 nm, 30-500 nm, 30-300 nm, 30-200 nm, 30-150 nm, 30-120 nm, 30-100 nm, 30-90 nm, 30-70 nm, 30-50 nm, 50-1000 nm, 50-800 nm, 50-500 nm, 50-300 nm, 50-200 nm, 50-150 nm, 50-120 nm, 50-100 nm, 50-90 nm, 50-70 nm, 70-1000 nm, 70-800 nm, 70-500 nm, 70-300 nm, 70-200 nm, 70-150 nm, 70-120 nm, 70-100 nm, 70-90 nm, 80-1000 nm, 80-800 nm, 80-500 nm, 80-300 nm, 80-200 nm, 80-150 nm, 80-120 nm, 80-100 nm, 80-90 nm, 90-1000 nm, 90-800 nm, 90-500 nm, 90-300 nm, 90-200 nm, 90-150 nm, 90-120 nm, 90-100 nm, 100-1000 nm, 100-800 nm, 100-500 nm, 100-300 nm, 100-200 nm, 100-150 nm, 100-120 nm, 120-1000 nm, 120-800 nm, 120-500 nm, 120-300 nm, 120-200 nm, 120-150 nm, 150-1000 nm, 150-800 nm, 150-500 nm, 150-300 nm, 150-200 nm, 200-1000 nm, 200-800 nm, 200-500 nm, or 200-300 nm, as determined by dynamic light scattering using e.g., a Malvern Zetasizer model, or nano ZS. In some embodiments, the nanoparticle composition of the present application comprises nanoparticles having an average particle diameter of 92-96 nm, 90-98 nm or 85-105 nm.

The lipophilic or hydrophobic component is dispersed in the polar liquid carrier to form a nanoemulsion that contains single layer micelles with a polar outer surface and an inner hydrophobic space filled with the lipophilic or hydrophobic component and/or other hydrophobic molecules, and double layer liposomes with a polar outer surface and an inner hydrophilic space. Because hydrophobic gases, such as oxygen and nitric oxide (NO), preferentially dissolve in the lipid core of the micelles relative to water or other aqueous environments, the nanoparticle composition of the present application provides the ability to carry oxygen and other hydrophobic gases to bodily tissues.

The solubility of hydrophobic gases in the lipophilic or hydrophobic core promotes the uptake and transport of these gases to tissues. The endogenously produced gases carbon monoxide, nitric oxide and hydrogen sulfide can also be carried in the emulsion for the modulation of the vascular tone and apoptotic processes.

In some embodiments, the nanoparticle composition is an oxygenated nanoparticle composition that enhances aerobic metabolism. In some embodiments, the nanoparticle composition is an oxygenated nanoparticle composition with an oxygen content of 1-50, 1-40, 1-30, 1-20, 1-10, 1-5, 1-2, 2-50, 2-40, 2-30, 2-20, 2-10, 2-5, 5-50, 5-40, 5-30, 5-20, 5-10, 10-50, 10-40, 10-30, 10-20, 15-50, 15-40, 15-30, 15-20, 20-50, 20-40, 20-30, 25-50, 25-40, 25-30, 30-50, 30-40 or 40-50 ml O₂/100 ml nanoparticle composition.

In some embodiments, the nanoparticle composition comprise NO in an amount of 1-50, 1-40, 1-30, 1-20, 1-10, 1-5, 1-2, 2-50, 2-40, 2-30, 2-20, 2-10, 2-5, 5-50, 5-40, 5-30, 5-20, 5-10, 10-50, 10-40, 10-30, 10-20, 15-50, 15-40, 15-30, 15-20, 20-50, 20-40, 20-30, 25-50, 25-40, 25-30, 30-50, 30-40 or 40-50 ml NO/100 ml nanoparticle composition.

Xenon and argon are hydrophobic gases that could provide protection of the brain in pathological states such as seizures. In some embodiments, the nanoparticle composition comprise Xe or Ar or both in an amount of 1-50, 1-40, 1-30, 1-20, 1-10, 1-5, 1-2, 2-50, 2-40, 2-30, 2-20, 2-10, 2-5, 5-50, 5-40, 5-30, 5-20, 5-10, 10-50, 10-40, 10-30, 10-20, 15-50, 15-40, 15-30, 15-20, 20-50, 20-40, 20-30, 25-50, 25-40, 25-30, 30-50, 30-40 or 40-50 ml O₂/100 ml nanoparticle composition.

In some embodiments, the nanoparticle composition of the present application further comprises inhibitors of apoptosis (e.g., Z-VAD-FMY, an apoptosis inhibiting peptide), protectors of mitochondrial integrity (e.g., Cyclosporin A, an inhibitor of mitochondrial inner pore opening), modulators of signal transduction, such as diacylglycerol or cyclic GMP, or an antioxidant, such as Coenzyme Q10.

When employing nanoparticles having liposomes with an average diameter below 30 nm, the liposomes can traverse the endothelial cell layer and enter the interstitial space. Such liposomes may be employed in situations where the permeability of the vascular space has not or has increased or to promote cellular absorption of lipophilic or hydrophobic mediators or to promote entry of molecules or cellular components that can favorably modulate intracellular mechanisms.

In certain cases, the nanoparticle composition of the present application is capable of exerting an osmotic force and/or absorb mediators of tissue injury, such as prostaglandins, nitric oxide, leukotrienes, and thromboxane, and other lipophilic or hydrophobic mediators such as platelet activating factors. Thus, in some cases, the nanoparticles of the present application are able to absorb toxic molecules produced by hyperprocalcitonemia patients. For example, lymph factors produced in the gut and thoracic duct lymph nodes may result in acute lung injury and red blood cell deformability. Other toxic molecules include, but are not limited to, leukotrienes, prostaglandins, nitric oxide, endotoxin and tumor necrosis factor (TNF). The nanoparticles in the nanoparticle composition allow effective absorption of lipophilic or hydrophobic chemical mediators. In some cases the absorption may be the result of electrostatic interactions. In other cases, the nanoparticles may be loaded with antagonists to toxic chemical mediators, such as antibodies to endotoxins.

In hyperprocalcitonemia patients having increased vascular wall permeability caused by e.g., capillary leak, the small size of the above-described phospholipid nanoparticles (PNs) facilitates their entry into interstitial spaces that would be otherwise restricted by larger structures. Capillary leak is caused by the death of endothelial cells and the actions of neutrophils. It is mediated by cytokines such as IL-1 and TNF as well as nitric oxide. Neutrophils adhere to damaged endothelial cells and release reactive oxygen species and cell wall damaging enzymes such as myeloperoxidase. The nanoparticles could get into the interstitium via the capillary leak and provide e.g., an anti-inflammatory effect within the interstitial space.

Preferably the nanoparticle compositions are formulated to comprise LMs and/or liposomes that are stable at room temperature (e.g., 25° C.) or 5° C. for a period of at least 3 days, 7 days, two weeks, 4 weeks, 12 weeks, 20 weeks, 180 days, 30 week, 40 weeks, one year or more. Stability may be determined by measuring the change in particle diameter. An unstable emulsion would have micelles that coalesce and form larger diameter micelles. In certain preferred embodiments, the nanoparticle composition is stable for at least 4 weeks at room temperature.

In some embodiments, the nanoparticle composition is formed from soybean oil in an amount of 5%-40% (w/v) and lecithin in an in an amount of 0.1%-18% (w/v). In some embodiments, the nanoparticle composition further comprises NaCl at a final concentration of 50-200 mM. In some embodiments, the nanoparticle composition further comprises glycerin in an amount of 1-5%. In one embodiment, the nanoparticle composition comprises 10% (w/v) soybean oil, 0.6% (w/v) egg lecithin, 1.13% (w/v) glycerin and 77 mM NaCl. In another embodiment, the nanoparticle composition comprises 20% (w/v) soybean oil, 1.2% (w/v) egg lecithin, and 2.25% (w/v) egg lecithin. In another embodiment, the nanoparticle composition comprises 20% (w/v) soybean oil, 1.2% (w/v) egg lecithin, and 2.25% (w/v) egg lecithin and 77 mM NaCl.

In some embodiments, the composition is formed from soybean oil in an amount of 10%-40% (w/v), preferably 15%-35%, lecithin in an in an amount of 1%-18% (w/v), preferably 10%-15%, sodium chloride and sodium lactate as electrolytes, wherein the total electrolyte composition is between 50 mM to 200 mM, histidine in an amount between 0.1 mM to 10 mM, and water such that the lecithin forms (1) lipid-carrying micelles having a lipophilic or hydrophobic core in an aqueous solution and the resulting micelles have an average diameter between 1-150 nm, preferably between 40 nm and 120 nm, as determined by dynamic light scattering, and are stable for at least 4 weeks at room temperature; and (2) liposomes having a diameter in the range of 1-25 nm, as determined by electron microscopy.

In another embodiment, the nanoparticle composition comprises 10-40% (w/v) soybean oil and 6-18% (w/v) egg lecithin or soybean lecithin. In some embodiments, the nanoparticle composition further comprises 0.6% (w/v) NaCl, 0.385% (w/v) Na(L) lactate, and 0.155% (w/v) histidine. In some embodiments, the nanoparticle composition comprises 20-30% (w/v) soybean oil, 12% (w/v) egg lecithin or soybean lecithin, 0.6% (w/v) NaCl, 0.385% (w/v) Na(L) lactate, and 0.155% (w/v) histidine.

In another embodiment, the nanoparticle composition comprises 20% (w/v) soybean oil, 12% (w/v) egg lecithin or soybean lecithin, 0.6% (w/v) NaCl, 0.385% (w/v) Na(L) lactate, and 0.155% (w/v) histidine, wherein the nanoparticle composition is prepared under conditions that form nanoparticles (including liposomes and micelles) with an average diameter of 250-300 nm, as measured by dynamic light scattering. In some embodiments, the nanoparticles comprise liposomes with diameters in the range of 1-25 nm or 7-20 nm, as measured by electronic microscopy and micelles with diameters in the range of 30-130 nm or 40-100 nm, as measured by electronic microscopy.

In another embodiment, the nanoparticle composition comprises 30% (w/v) soybean oil, 12% (w/v) egg lecithin or soybean lecithin, 0.6% (w/v) NaCl, 0.385% (w/v) Na(L) lactate, and 0.155% (w/v) histidine, wherein the nanoparticle composition is prepared under conditions that form nanoparticles (including liposomes and micelles) with an average diameter of 80-120 nm, as measured by dynamic light scattering. In some embodiments, the nanoparticles comprise liposomes with diameters in the range of 1-25 nm or 7-20 nm, as measured by electronic microscopy and micelles with diameters in the range of 30-130 nm or 40-100 nm, as measured by electronic microscopy.

Other oils, such as oil from chia beans, pumpkin seeds or other sources may be used. In certain embodiments, the above described nanoparticle composition may further comprise about 2-40% (w/v), about 2-20% (w/v), about 4-10% (w/v), or about 5% (w/v) albumin or albumin polymers or albumin polymers conjugated with amino acids or peptides, which are added to the nanoparticle composition after the formation of micelles. In other embodiment the hydrophobic or the hydrophilic component is carried within erythrocyte ghosts.

In certain embodiments, the LMs make up 10-40% (w/w) of the nanoparticle composition, while the liposomes make up 5-30% (w/w) of the nanoparticle composition. In some embodiments, the LMs are made using soybean oil and the liposomes are made using chia bean oil, which has a greater anti-inflammatory effect than that of soybean oil.

In certain embodiments the nanoparticle composition of the present application comprises a lipophilic or hydrophobic component selected from the group consisting of soybean oil, chia bean oil and algae oil, an emulsifier selected from the group consisting of phospholipids and α-phosphatidylcholine, and an amino acid or n-acetyl amino acid at a final concentration of 0.2-20 mM, 0.5-10 mM, 0.5-5 mM or 0.5-2 mM.

In certain embodiments, the nanoparticle composition has a final amino acid concentration of 0.000001-10 mM, 0.01-10 mM, 0.1-10 mM, 0.2-10 mM, 0.5-10 mM, 1-10 mM, 2.5-10 mM, 5-10 mM or 7.5-10 mM. In certain embodiments, the nanoparticle composition has a final amino acid concentration of 0.001, 0.01, 0.1, 0.2, 0.5, 1, 2.5, 5, 7.5, or 10 mM. The emulsifier: lipophilic or hydrophobic component ratio (w/w) may range between about 1:400 to about 1:1, preferably between about 1:200 to about 1:50. In one embodiment, the emulsifier: lipophilic or hydrophobic component ratio (w/w) is about 1:100. In another embodiment, the emulsifier: lipophilic or hydrophobic component ratio (w/w) is about 1.2:100.

In some embodiments, the nanoparticle composition substantially consists of liposomes and do not include a lipophilic or hydrophobic component, such as soybean oil.

In some embodiments, the nanoparticle composition includes one or more active pharmaceutical ingredients or agents (e.g., nucleic acids, proteins, small molecule drugs etc.) into the LMs and/or liposomes. The active pharmaceutical ingredients or agents can be incorporated into the lipophilic or hydrophobic core of LMs or liposomes or into the hydrophilic core of liposomes.

In one embodiment, the nanoparticle composition comprises soybean oil, egg phospholipids and an amino acid, beta-endorphin or other modulator that acts at femtomolar concentrations or higher at a final concentration of 0.1 femtomolar (fM) to 10 mM.

The nanoparticle compositions of the present application are free of hemoglobin, derivatives of hemoglobin, perfluorocarbon and derivatives of perfluorocarbon. As used herein, a composition is “free of hemoglobin, derivatives of hemoglobin, perfluorocarbon and derivatives of perfluorocarbon” if the composition does not contain any hemoglobin, derivatives of hemoglobin, perfluorocarbon and derivatives of perfluorocarbon, or if the composition contains hemoglobin, derivatives of hemoglobin, perfluorocarbon and derivatives of perfluorocarbon at levels below 0.1% w/v.

The nanoparticle compositions of the present application are typically free of Ca⁺⁺, K⁺, and Mg⁺⁺. and added Al⁺⁺⁺ In certain embodiments, Ca⁺⁺ and K⁺ are added to the nanoparticle composition just prior to use (e.g., within 24 hours prior to use). In other embodiments Ca⁺⁺ is premixed with the nanoparticle composition Because Al⁺⁺⁺ is toxic to bone, brain, hematopoiesis, heme synthesis, globulin synthesis, iron absorption and metabolism, and fetal development, all oils and other components must have the minimum amount of Al⁺⁺⁺ possible. In certain embodiments, the nanoparticle composition contains Al⁺⁺⁺ at a concentration of less than 25 mg/1, 20 mg/1, 10 mg/l or 5 mg/l. In other embodiments, the nanoparticle composition is free of Al⁺⁺⁺, i.e., undetectable by conventional methods.

In certain embodiments, the micelles in the nanoparticle composition of the present application are free-moving micelles that are not encapsulated in any type of particles. Further, the wall of the micelles is comprised of either a single layer or a double layer of the amphiphilic emulsifier molecules so that the micelles may easily merge with the cell membrane of the tissue that comes in contact with the nanoparticle composition. Further, the micelles in the nanoparticle composition of the present application are free of hemoglobin, derivatives of hemoglobin, perfluorocarbon and derivatives of perfluorocarbon.

### Amphiphilic Emulsifier

The amphiphilic emulsifier can be any amphiphile or amphiphilic molecule that will have its hydrophobic tail in the lipophilic or hydrophobic core of the micelle and its hydrophilic end in contact with the polar carrier.

As used herein, the term “amphiphile” refers to a chemical compound possessing both hydrophilic and lipophilic or hydrophobic properties. Examples of amphiphiles include, but are not limited to, naturally-occurring amphiphiles such as phospholipids, cholesterol, glycolipids, fatty acids, bile acids, and saponins; and synthetic amphiphiles such as amphiphilic peptides.

Examples of phospholipids include natural or synthetic phospholipids such as phosphatidylcholine, phosphatidylethanolamine, phosphatidylserine, phosphatidic acid, phosphatidylglycerol, phosphatidylinositol, lisophosphatidylcholine, sphingomyelin, egg yolk lecithin, soybean lecithin, and a hydrogenated phospholipid.

Examples of glycolipids include glyceroglycolipids and sphingoglycolipids. Examples of glyceroglycolipids include digalactosyl diglycerides (such as digalactosyl dilauroyl glyceride, digalactosyl dimyristoyl glyceride, digalactosyl dipalmitoyl glyceride, and digalactosyl distearoyl glyceride) and galactosyl diglycerides (such as galactosyl dilauroyl glyceride, galactosyl dimyristoyl glyceride, galactosyl dipalmitoyl glyceride, and galactosyl distearoyl glyceride). Examples of sphingoglycolipids include galactosyl cerebroside, lactosyl cerebroside, and ganglioside.

Examples of the sterols include cholesterol, cholesterol hemisuccinate, 3β-[N—(N′, N′-dimethylaminoethane)carbamoyl]cholesterol, ergosterol, and lanosterol.

In one embodiment, the emulsifier comprises egg phospholipid or egg yolk lecithin. In another embodiment, the emulsifier is soybean lecithin or alpha-phosphatidylcholine.

In other embodiments, the emulsifier may constitute between 0.1-100%, 0.1-90%, 0.1-80%, 0.1-70%, 0.1-60%, 0.1-50%, 0.1-40%, 0.1-30%, 0.1-20%, 0.1-15%, 0.1-10%, 0.1-5%, 0.1-2%, 0.3-100%, 0.3-90%, 0.3-80%, 0.3-70%, 0.3-60%, 0.3-50%, 0.3-40%, 0.3-30%, 0.3-20%, 0.3-15%, 0.3-10%, 0.3-5%, 0.3-2%, 0.6-100%, 0.6-90%, 0.6-80%, 0.6-70%, 0.6-60%, 0.6-50%, 0.6-40%, 0.6-30%, 0.6-20%, 0.6-15%, 0.6-10%, 0.6-5%, 0.6-2%, 2-100%, 2-90%, 2-80%, 2-70%, 2-60%, 2-50%, 2-40%, 2-30%, 2-20%, 2-15%, 2-10%, 2-5%, 6-100%, 6-90%, 6-80%, 6-70%, 6-60%, 6-50%, 6-40%, 6-30%, 6-20%, 6-15%, 6-10%, 10-100%, 10-90%, 10-80%, 10-70%, 10-60%, 10-50%, 10-40%, 10-30%, 10-20%, 10-15%, 15-100%, 15-90%, 15-80%, 15-70%, 15-60%, 15-50%, 15-40%, 15-30%, 15-20%, 20-100%, 20-90%, 20-80%, 20-70%, 20-60%, 20-50%, 20-40%, 20-30%, 30-100%, 30-90%, 30-80%, 30-70%, 30-60%, 30-50%, 30-40%, 40-100%, 40-90%, 40-80%, 40-70%, 40-60%, 40-50%, 50-100%, 50-90%, 50-80%, 50-70%, 50-60% (w/v or v/v) of the nanoparticle composition. In some embodiments, the emulsifier may constitute greater than 1%, 2%, 5%, 10%, 15%, 20%, 25%, 30%, 35%, 40%, 45%, 505, 55%, 60%, 65%, 70%, 75%, 80%, 85%, 90% or 95% (w/v or v/v) of the nanoparticle composition. In certain embodiments, the emulsifier is present at a level of about 1%, 6%, about 7%, about 8%, about 9%, about 10%, about 11%, about 12%, about 13%, about 14%, about 15%, about 16%, about 18%, about 20% (w/v or v/v) of the nanoparticle composition or any other range between any two of these listed integers. In other embodiments, the emulsifier is present at a level of about 7-9%, 9-11%, 11-13%, 13-15%, 15-17%, 17-19%, 10-14%, 9-15%, or 8-16% (w/v or v/v) of the nanoparticle composition or any other range between any two of these listed integers. In yet other embodiments, the upper limit and/or lower limit of the emulsifier is defined by any of the listed concentrations described herein.

In certain preferred embodiments, the emulsifier is a lecithin, such as egg yolk lecithin or soybean lecithin in one of the above described amounts or ranges.

### Lipophilic or Hydrophobic Component

As used herein, the term “lipophilic component” refers to a fat-soluble material that is naturally occurring, or non-naturally occurring. Examples of lipophilic components include but are not limited to, fatty acyls, glycerolipids, phospholipids, sphingolipids, sterol lipids, prenol lipids, saccharolipids, polyketides, non-natural lipid(s), cationic lipid(s), amphipathic alkyl amino acid derivative, adialkyldimethylammonium, polyglycerol alkyl ethers, polyoxyethylene alkyl ethers, tri-n-octylamine, boric acid, tris(3,5-dimethyl-4-heptyl) ester, triglycerides, diglycerides and other acylglycerols, such as tetraglycerol, pentaglycerol, hexaglycerol, heptaglycerol, octoglycerol, nonaglyceol and decaglycerol, hydrophobic peptides, hydrophobic polysaccharides, silicones, lipopeptides, cyclopeptides and mixtures thereof. In certain embodiments, the lipophilic or hydrophobic component comprises soybean oil, chia bean oil or algae oil.

In one embodiment, the hydrophobic component is soybean oil. The Hydrophobic component may also be derived from chia beans that have a high concentration of anti-inflammatory omega 3 fatty acids. Soybean oil is thrombogenic and procoagulant, and therefore would be preferred when clotting is desired. After bleeding is no longer an issue, oils rich in omega 3 fatty acids would be favored because of their anti-thromobogenic properties. Oils rich in omega 3 fatty acids include, but are not limited to chia oil, algae oil, pumpkin oil, flaxseed oil or fish oil.

In certain embodiments, the lipophilic or hydrophobic component comprises an unsaturated fatty acid with one or more alkenyl functional groups in a cis or trans configuration. A cis configuration means that adjacent hydrogen atoms or other groups are on the same side of the double bond. In a trans configuration these moieties are on different sides of the double bond. The rigidity of the double bond freezes its conformation and, in the case of the cis isomer, causes the chain to bend and restricts the conformational freedom of the fatty acid. In general, the more double bonds the chain has, the less flexibility it has. When a chain has many cis bonds, it becomes quite curved in its most accessible conformations. For example, oleic acid, with one double bond, has a “kink” in it, while linoleic acid, with two double bonds, has a more pronounced bend. Alpha-linolenic acid, with three double bonds, favors a hooked shape. The effect of this is that in restricted environments, such as when fatty acids are part of a phospholipid in a lipid bilayer, or triglycerides in lipid droplets, cis bonds limit the ability of fatty acids to be closely packed and therefore could affect the melting temperature of the membrane or of the fat. In some embodiments, the lipophilic or hydrophobic component comprises up to 1%, 2%, 5%, 10%, 15%, 20%, 25%, 30%, 35%, 40%, 45%, 50%, 55%, 60%, 65%, 70%, 75%, 80%, 85%, 90%, 95% or 99% (w/v or v/v) unsaturated fatty acid(s) that have one or more alkenyl functional groups in cis configuration.

Examples of cis-unsaturated fatty acids include, but are not limited to, obtusilic acid, linderic acid, tsuzuic acid, palmito-oleic acid, oleic acid, elaidic acid, vaccenic acid, petroselinic acid, gadoleic acid, eicosenoic acid, erucic acid, cetoleic acid, nervonic acid, ximenic acid and lumepueic acid; n-3 type unsaturated fatty acids such as α-linolenic acid, stearidonic acid, eicosatetraenoic acid, eicosapentaenoic acid, docosapentaenoic acid and docosahexaenoic acid; n-6 type unsaturated fatty acids such as linoleic acid, linoelaidic acid, γ-linolenic acid, bis-homo-γ-linolenic acid and arachidonic acid; conjugated fatty acids such as conjugated linoleic acid and α-eleostearic acid; fatty acids carrying double bonds at the 5-position thereof such as pinolenic acid, sciadonic acid, juniperic acid and columbinic acid; polyvalent unsaturated fatty acids, other than those listed above, such as hiragonic acid, moroctic acid, clupanodonic acid and nishinic acid; branched fatty acids such as isobutyric acid, isovaleric acid, iso acid and anti-iso acid; hydroxy fatty acids such as α-hydroxy acid, β-hydroxy acid, mycolic acid and polyhydroxy acid; epoxy-fatty acids; keto-fatty acids; and cyclic fatty acids. In certain embodiments, the lipophilic or hydrophobic component also comprises amphiphilic molecules.

The lipophilic or hydrophobic component may constitute about 0-80%, 1-80%, 1-70%, 1-60%, 1-50%, 1-40%, 1-30%, 1-20%, 5-80%, 5-70%, 5-60%, 5-50%, 5-40%, 5-30%, 5-20%, 10-80%, 10-70%, 10-60%, 10-50%, 10-40%, 10-30%, 10-20%, 15-80%, 15-70%, 15-60%, 15-50%, 15-40%, 15-30%, 15-20%, 20-80%, 20-70%, 20-60%, 20-50%, 20-40%, 20-30%, 30-80%, 30-70%, 30-60%, 30-50%, 30-40%, 40-80%, 40-70%, 40-60%, 40-50%, 40-80%, 40-70%, 40-60%, 40-30%, 50-80%, 50-70%, 50-60%, 60-80%, 60-70% or 70-80% (w/v or v/v) of the nanoparticle composition. In certain embodiments, the lipophilic or hydrophobic component constitutes about 10%, about 15%, about 20%, about 25%, about 30% and about 35% (w/v or v/v) of the nanoparticle composition. In some embodiments, the lipophilic or hydrophobic component comprises between 0-35%, 5-35%, 10-35%, 15-35%, 20-35%, 25-35%, 30-35%, 0-30%, 5-30%, 10-30%, 15-30%, 20-30%, 25-30%, 0-25%, 5-25%, 10-25%, 15-25%, 20-25%, 0-15%, 5-15% or 10-15% (w/v or v/v) of the nanoparticle composition, or any percent range combination comprising integer values selected from the group consisting of 10%, 15%, 20%, 25%, 30% or 35% (w/v or v/v). In yet other embodiments, the upper limit and/or lower limit of the lipophilic or hydrophobic component is defined by any of the listed concentrations described herein. Polar liquid carrier

The polar liquid carrier can be any pharmaceutically acceptable polar liquid that is capable of forming an emulsion with the lipid. The term “pharmaceutically acceptable” refers to molecular entities and compositions that are of sufficient purity and quality for use in the formulation of a composition or medicament of the present application and that, when appropriately administered to an animal or a human, do not produce an adverse, allergic or other untoward reaction. Since both human use (clinical and over-the-counter) and veterinary use are equally included within the scope of the present application, a pharmaceutically acceptable formulation would include a composition or medicament for either human or veterinary use. In one embodiment, the polar liquid carrier is water or a water based solution. In another embodiment, the polar liquid carrier is a non-aqueous polar liquid such as dimethyl sulfoxide, polyethylene glycol and polar silicone liquids.

A water-based solution generally comprises a physiologically compatible electrolyte vehicle isosmotic or near isosmotic with whole blood. The carrier can be, for example, physiological saline, a saline-glucose mixture, Ringer's solution, lactated Ringer's solution, Locke-Ringer's solution, Krebs-Ringer's solution, Hartmann's balanced saline, heparinized sodium citrate-citric acid-dextrose solution, and polymeric plasma substitutes, such as polyethylene oxide, polyvinyl pyrrolidone, polyvinyl alcohol and ethylene oxide-propylene glycol condensates. The nanoparticle composition may additionally comprise other constituents such as pharmaceutically-acceptable carriers, diluents, fillers and salts, the selection of which depends on the dosage form utilized, the condition being treated, the particular purpose to be achieved according to the determination of the ordinarily skilled artisan in the field and the properties of such additives.

### Electrolytes

In one embodiment, the nanoparticle composition of the present application includes one or more electrolytes. The electrolyte to be used in the present application typically includes various electrolytes to be used for medicinal purposes. Examples of the electrolyte include sodium salts (e.g., sodium chloride, sodium hydrogen carbonate, sodium citrate, sodium lactate, sodium sulfate, sodium dihydrogen phosphate, disodium hydrogen phosphate, sodium acetate, sodium glycerophosphate, sodium carbonate, an amino acid sodium salt, sodium propionate, sodium hydroxybutyrate, and sodium gluconate), potassium salts (e.g., potassium chloride, potassium acetate, potassium gluconate, potassium hydrogen carbonate, potassium glycerophosphate, potassium sulfate, potassium lactate, potassium iodide, potassium dihydrogen phosphate, dipotassium hydrogen phosphate, potassium citrate, an amino acid potassium salt, potassium propionate, and potassium hydroxybutyrate), calcium salts (e.g., calcium chloride, calcium gluconate, calcium lactate, calcium glycerophosphate, calcium pantothenate, and calcium acetate), magnesium salts (e.g., magnesium chloride, magnesium sulfate, magnesium glycerophosphate, magnesium acetate, magnesium lactate, and an amino acid magnesium salt), ammonium salts (e.g., ammonium chloride), zinc salts (e.g., zinc sulfate, zinc chloride, zinc gluconate, zinc lactate, and zinc acetate), iron salts (e.g., iron sulfate, iron chloride, and iron gluconate), copper salts (e.g., copper sulfate), and manganese salts (for example, manganese sulfate). Among those, particularly preferable are sodium chloride, potassium chloride, magnesium chloride, disodium hydrogen phosphate, dipotassium hydrogen phosphate, potassium dihydrogen phosphate, sodium lactate, sodium acetate, sodium citrate, potassium acetate, potassium glycerophosphate, calcium gluconate, calcium chloride, magnesium sulfate, and zinc sulfate.

Concentrations of calcium, sodium, magnesium or potassium ions are typically within the range of normal physiological concentrations of such ions in plasma. In general, the desired concentration of these ions is obtained from the dissolved chloride salts of calcium, sodium and magnesium. The sodium ions may also come from a dissolved organic salt of sodium that is also in solution.

In one embodiment, the electrolytes comprise sodium chloride, sodium lactate or both.

In a particular embodiment, the nanoparticle composition comprises sodium chloride at a percent concentration of about 0.2-1%, 0.3-0.9%, 0.4-0.8%, 0.5-0.7% or about 0.6% (w/v).

In another embodiment, the nanoparticle composition comprises sodium chloride at a concentration of 50-150 mM, 70-130 mM, 80-120 mM, 90-110 mM, 95-100 mM, or about 97.4 mM.

In another embodiment, the nanoparticle composition comprises sodium L-lactate, sodium D-lactate or a mixture thereof at a percent concentration of about 0.1-0.7%, 0.2-0.6%, 0.3-0.5%, 0.35-0.45%, 0.38-0.39% or about 0.385% (w/v).

In another embodiment, the nanoparticle composition comprises sodium L-lactate, sodium D-lactate or a mixture thereof at a concentration of 10-60 mM, 20-50 mM, 30-40 mM or about 34 mM.

In one embodiment, the sodium ion concentration is in a range from about 70-180 mM, 90-170 mM, 70-160 mM, 100-160 mM, 110-150 mM, 120-140 mM, 125-135 mM, 131-133 mM or about 131.4 mM.

In one embodiment, the concentration of calcium ion is in a range of about 0.5-4.0 mM, 0.5-1.0 mM, 0.5-2 mM, 0.5-3 mM, 1-2 mM, 1-3 mM, 1-4 mM, 2-2.5 mM, 2-3 mM, 2-4 mM, 2.5-3 mM or 3-4 mM.

In one embodiment, the concentration of magnesium ion is in a range of 0 to 10 mM. In another embodiment, the concentration of magnesium ion is in a range of about 0.3-0.45 mM, 0.3-0.35 mM, 0.3-0.4 mM, 0.35-0.4 mM, 0.35-0.4 mM, 0.35-0.4 mM or 0.4-0.45 mM. It is best not to include excessive amounts of magnesium ion in the nanoparticle composition of the invention because high magnesium ion concentrations negatively affect the strength of cardiac contractile activity. In a preferred embodiment of the invention, the nanoparticle composition contains subphysiological amounts of magnesium ion.

In one embodiment, the concentration of potassium ion is in a subphysiological range of between 0-5 mEq/1 K+ (0-5 mM), preferably 2-3 mEq/1 K+ (2-3 mM). Thus, the nanoparticle composition allows for dilution of the potassium ion concentration in stored transfused blood. As a result, high concentrations of potassium ion and potential cardiac arrhythmias and cardiac insufficiency caused thereby can be more easily controlled. The nanoparticle composition containing a subphysiological amount of potassium is also useful for purposes of blood substitution and low temperature maintenance of a subject.

In one embodiment, the concentration of chloride ion is in the range of 50-200 mM, 50-150 mM, 70-180 mM, 70-130 mM, 80-170 mM, 80-120 mM, 90-160 mM, 90-110 mM, 95-150 mM, 95-100 mM, or about 97.4 mM. In another embodiment, the concentration of chloride ion is in the range of 110 mM to 125 mM.

Other sources of ions include sodium salts (e.g., sodium hydrogen carbonate, sodium citrate, sodium lactate, sodium sulfate, sodium dihydrogen phosphate, disodium hydrogen phosphate, sodium acetate, sodium glycerophosphate, sodium carbonate, an amino acid sodium salt, sodium propionate, sodium 3-hydroxybutyrate, and sodium gluconate), potassium salts (e.g., potassium acetate, potassium gluconate, potassium hydrogen carbonate, potassium glycerophosphate, potassium sulfate, potassium lactate, potassium iodide, potassium dihydrogen phosphate, dipotassium hydrogen phosphate, potassium citrate, an amino acid potassium salt, potassium propionate, and potassium 3-hydroxybutyrate), calcium salts (e.g., calcium gluconate, calcium lactate, calcium glycerophosphate, calcium pantothenate, and calcium acetate), magnesium salts (e.g., magnesium sulfate, magnesium glycerophosphate, magnesium acetate, magnesium lactate, and an amino acid magnesium salt), ammonium salts, zinc salts (e.g., zinc sulfate, zinc chloride, zinc gluconate, zinc lactate, and zinc acetate), iron salts (e.g., iron sulfate, iron chloride, and iron gluconate), copper salts (e.g., copper sulfate), and manganese salts (for example, manganese sulfate). Among those, particularly preferable are sodium chloride, potassium chloride, magnesium chloride, disodium hydrogen phosphate, dipotassium hydrogen phosphate, potassium dihydrogen phosphate, sodium lactate, sodium acetate, sodium citrate, potassium acetate, potassium glycerophosphate, calcium gluconate, calcium chloride, magnesium sulfate, choline chloride and zinc sulfate.

### Gas Carrying Capacity of the Nanoparticle Composition

The lipophilic or hydrophobic component in the nanoparticle composition, in forms such as micelles and/or erythrocyte ghosts provides the ability for the nanoparticle composition to carry a larger amount of lipophilic gases than of a purely aqueous solution. Specifically, the lipophilic gases are dissolved into the lipophilic portion of the nanoparticle composition to form a homogeneous solution with the lipophilic or hydrophobic component and any other hydrophobic liquid material that may be present in the lipophilic or hydrophobic portion of the nanoparticle composition.

In one embodiment, the lipophilic gas is oxygen. Oxygen is 4.41 times more soluble in lipid than in water (Battion et al., J. Amer. Oil Chem. Soc. 1968, 45:830-833). Accordingly, a nanoparticle composition with a higher lipid content would be able to carry more oxygen than a nanoparticle composition with a lesser lipid content. In one embodiment, the nanoparticle composition has a lipid content of about 1-80% (w/v). In other embodiments, the nanoparticle composition has a lipid content of about 10-80% (w/v), 20-60% (w/v), about 20-50% (w/v), about 20-40% (w/v) or about 20-25% (w/v). In yet another embodiment, the nanoparticle composition has a lipid content of about 21.8%. In certain embodiments, the nanoparticle composition is prepared by mixing the lipophilic or hydrophobic component and the polar liquid component in the presence of regular air. In other embodiment, the nanoparticle composition is further oxygenated by bubbling regular air or pure oxygen through the nanoparticle composition for a desired period of time. Since bubbles are undesirable in the circulation due to the possibility of air embolization a bubble trap would have to be added to remove bubbles leaving only the gas that has been solubilized in the core of the micelle, in the polar carrier or attached to proteins or other additives. The gas may also be loaded onto the micelles by equilibration of the micelles with an atmosphere enriched with the gas combined with gentle movement of the nanoparticle composition in a mixture chamber in order to avoid the creation of bubbles. Loading may also be done under pressures greater than 1 atmosphere followed by release of the pressure to allow the release of excess gas.

In another embodiment, the lipophilic gas is xenon (Xe) or argon (Ar). In another embodiment, the lipophilic gas is nitric oxide (NO). In another embodiment, the lipophilic gas is hydrogen sulfide (H₂S). In yet another embodiment, the lipophilic gas is carbon monoxide (CO).

In one embodiment, the nanoparticle composition contains micelles loaded with a gas mixture (e.g., a mixture of oxygen, hydrogen sulfide, carbon monoxide and/or nitric oxide). In another embodiment, the nanoparticle composition contains a mixture of micelles loaded with various gases. For example, the mixture of micelles may contain 50% NO-loaded micelles and 50% O₂-loaded micelles.

### Rigid Nonplanar Molecules

The nanoparticle composition may further comprise molecules with a rigid nonplanar structure. Such molecules will create greater irregularity and more space for gas molecules in the hydrophobic core of the micelle structure, thereby modifying the gas carrying capacity of the micelles. Examples of such molecules include, but are not limited to, (+) naloxone, (+) morphine, and (+) naltrexone.

In one embodiment, molecules with a rigid nonplanar structure is (+) naloxone which, unlike the opiate receptor antagonist (−) naloxone, does not bind to opiate receptor and will not increase pain as (−) naloxone would. In another embodiment, (+) naloxone is used at a concentration of 10⁻⁵-10⁻⁴ M. In another embodiment, (+) naloxone is used at a concentration of 10⁻⁴ M or higher.

In one embodiment, (+) naloxone is used at a concentration range that produces anti-inflammatory effect at 10⁻⁵-10⁻⁴ M.

Molecules with a nonplanar structure also include organic molecules with branched structures. Examples of such molecules include, but are not limited to, tri-n-octylamine, tri-n-hexylamine, boric acid, tris(3,5,-dimethyl-4-heptyl) ester, metal complexed and non-metal complexed deuteroporphyrin dimethyl esters and their derivatives, hexaphenylsilole, and silicone polymers.

### Plasma Component

The nanoparticle composition may further comprise a plasma component. In one embodiment, the plasma is an animal plasma. In another embodiment, the plasma is human plasma. Although not wishing to be bound by any particular scientific theory, it is believed that the intravascular administration of fluids may dilute the concentration of coagulation factors to an undesirable level. Accordingly, using plasma as the diluent for the oxygen carrying component avoids this problem. Plasma can be collected by any means known in the art, provided that red cells, white cells and platelets are essentially removed. Preferably, it is obtained using an automated plasmapheresis apparatus. Plasmapheresis apparatuses are commercially available and include, for example, apparatuses that separate plasma from the blood by ultrafiltration or by centrifugation. An ultrafiltration-based plasmapheresis apparatus such as manufactured by Auto C, A200 (Baxter International Inc., Deerfield, IL) is suitable because it effectively removes red cells, white cells and platelets while preserving coagulation factors.

Plasma may be collected with an anticoagulant, many of which are well known in the art. Preferred anti-coagulants are those that chelate calcium such as citrate. In one embodiment, sodium citrate is used as an anticoagulant at a final concentration of 0.2-0.5%, preferably 0.3-0.4%, and most preferably at 0.38%. The plasma may be fresh, frozen, pooled and/or sterilized. While plasma from exogenous sources may be preferred, it is also within the present application to use autologous plasma that is collected from the subject prior to formulation and administration of the nanoparticle composition.

In addition to plasma from natural sources, synthetic plasma may also be used. The term “synthetic plasma,” as used herein, refers to any aqueous solution that comprises at least one plasma protein. Proteins resembling plasma protein may also be used.

### Oncotic Agent

In one embodiment, the nanoparticle composition further contains an oncotic agent in addition to the nanoparticles (micelles or liposomes). The oncotic agent is comprised of molecules whose size is sufficient to prevent their loss from the circulation by traversing the fenestrations of the capillary bed into the interstitial spaces of the tissues of the body. Examples of oncotic agents include, but are not limited to, dextran (e.g., a low-molecular-weight dextran), dextran derivatives (e.g., carboxymethyl dextran, carboxydextran, cationic dextran, and dextran sulfate), hydroxyethyl starch, hydroxypropyl starch, branched, unsubstituted or substituted starch, gelatin (e.g., modified gelatin), albumin (e.g., human plasma, human serum albumin, heated human plasma protein, and recombinant human serum albumin), PEG, polyvinyl pyrrolidone, carboxymethylcellulose, acacia gum, glucose, a dextrose (e.g., glucose monohydrate), oligosaccharides (e.g., oligosaccharide), a polysaccharide degradation product, an amino acid, and a protein degradation product. Among those, particularly preferable are low-molecular-weight dextran, hydroxyethyl starch, modified gelatin, and recombinant albumin.

Because of its antioxidant effects, albumin may also be used to minimize reactive oxygen species interaction with the components of the micelle and may also stabilize the micelle structure. In one embodiment, the oncotic agent is about 2%, 5%, 7% or 10% (w/v) albumin. In another embodiment, the oncotic agent is a polysaccharide, such as Dextran, in a molecular weight range of 30,000 to 50,000 daltons (D). In yet another embodiment, the oncotic agent is a polysaccharide, such as Dextran, in a molecular weight range of 50,000 to 70,000 D. High molecular weight dextran solutions are more effective in preventing tissue swelling due to their lower rates of leakage from capillaries.

In one embodiment, the concentration of the polysaccharide is sufficient to achieve (when taken together with chloride salts of sodium, calcium and magnesium, organic ion from the organic salt of sodium and hexose sugar discussed above) colloid osmotic pressure approximating that of normal human serum, about 28 mm Hg.

In another embodiment the oncotic agent is glycerol or mannitol in an amount of about 2%, 2.5%, 3%, 3.5%, 4%, 4.5%, 5%, 10%, 15%, 20%, 25% or 30% (w/v) of the nanoparticle composition. In other embodiments, nanoparticle composition comprises glycerol or mannitol in an amount of 2-5% w/v.

### Crystalloid Agent

The nanoparticle composition may also comprise a crystalloid agent. The crystalloid agent can be any crystalloid which, in the form of the nanoparticle composition, is preferably capable of achieving an osmolarity greater than 800 mOsm/l, i.e. it makes the nanoparticle composition “hypertonic”. Examples of suitable crystalloids and their concentrations in the nanoparticle composition include, but are not limited to, 3% w/v NaCl, 7% NaCl, 7.5% NaCl, and 7.5% NaCl in 6% w/v dextran. In one embodiment, the nanoparticle composition has an osmolarity of between 800 and 2400 mOsm/l.

### Anti-Inflammatory and Immunomodulatory Agent

In one embodiment, the nanoparticle composition of the present application further includes an anti-inflammatory or immunomodulatory agent. Examples of the anti-inflammatory agent shown to inhibit reactive oxygen species including, but are not limited to, histidine, albumin, (+) naloxone, prostaglandin D2, molecules of the phenylalkylamine class. Other anti-inflammatory compounds and immunomodulatory drug include interferon; interferon derivatives comprising betaseron, β-interferon; prostane derivatives comprising iloprost, cicaprost; glucocorticoids comprising cortisol, prednisolone, methyl-prednisolone, dexamethasone; immunsuppressives comprising cyclosporine A, methoxsalene, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; anti-TNF-antibodies; soluble receptors of interleukins or other cytokines; antibodies against receptors of interleukins or other cytokines, T-cell-proteins; and calcipotriols and analogues thereof taken either alone or in combination.

### Carbohydrates and Amino Acids

The nanoparticle composition may contain a carbohydrate or a mixture of carbohydrates. Suitable carbohydrates include, but are not limited to, simple hexose (e.g., glucose, fructose and galactose), mannitol, sorbitol or others known to the art. In one embodiment, the nanoparticle composition includes physiological levels of a hexose. “Physiological levels of a hexose” includes a hexose concentration of between 2 mM to 50 mM. In one embodiment, the nanoparticle composition contains 5 mM glucose. At times, it is desirable to increase the concentration of hexose in order to provide nutrition to cells. Thus, the range of hexose may be expanded up to about 50 mM if necessary to provide minimal calories for nutrition.

Other suitable carbohydrates include various saccharides to be used for medicinal purposes. Examples of the saccharides include xylitol, dextrin, glycerin, sucrose, trehalose, glycerol, maltose, lactose, and erythritol.

The nanoparticle composition may contain one or more amino acids and/or one or more oligopeptides. Suitable amino acids include, but are not limited to, alanine, arginine, aspartate, asparagine, cysteine, glutamate, glutamine, glycine, histidine, isoleucine, leucine, lysine, methionine,phenylalanine, proline, serine, threonine, tryptophan, tyrosine, threonine, tryptophan, valine and 2-aminopentaenoic acid. In one embodiment, the amino acid is selected from the group consisting of histidine, tyrosine, phenylalanine and cysteine. In another embodiment, the nanoparticle composition comprises one or more amino acids known to prevent apoptosis. Examples of such amino acids include glutamine, glycine, proline and 2-aminopentaenoic acid.

The amino acid may be used in the concentration range of 0.1 fM-200 mM, 0.1 fM-100 μM, 100 μM-10 nM, 10 nM-10 μM, 0.01-200 mM, 0.2-50 mM, or 0.5-2 mM. In one embodiment, the amino acid is used at a concentration of 1 mM.

### Buffering Agent

The nanoparticle composition of the present application may further comprise a biological buffer to maintain the pH of the fluid at the physiological range of pH7-8. Examples of biological buffers include, but are not limited to, N-2-Hydroxyethylpiperazine-N′-2-hydroxypropanesulfonic acid (HEPES), 3-(N-Morpholino)propanesulfonic acid (MOPS), 2-([2-Hydroxy-1,1-bis(hydroxymethyl)ethyl]amino)glyci ethanesulfonic acid (TES), 3-[N-tris(Hydroxy-methyl)methylamino]-2-hydroxyethyl]-1-piperazinepropanesulfonic acid (EPPS), Tris [hydrolymethyl]-aminoethane (THAM), and Tris [Hydroxylmethyl]methyl aminomethane (TRIS).

In one embodiment, the buffering agent is histidine, imidazole, substituted histidine or imidazole compounds retaining the amphoteric site of the imidazole ring, oligopeptides containing histidine or glycine (such as glygly) or mixtures thereof. Histidine is also capable of reducing reactive oxygen species and inhibiting cell shrinkage. (see e.g., Simpkins et al., J Trauma. 2007, 63:565-572). Histidine or imidazole may be used at a concentration of about 1 Mm, 5 Mm, 10 Mm, 20 Mm, 30 Mm, 40 Mm, 50 Mm or in a concentration range of about 0.1 Mm to about 200 Mm, 1 Mm to about 100 Mm, 5 Mm to about 50 Mm, 5 Mm to about 20 Mm or any other range between any of the histidine concentrations listed herein.

In another embodiment, the nanoparticle composition of the present application uses normal biological components to maintain in vivo biological pH. Briefly, some biological compounds, such as lactate, are capable of being metabolized in vivo and act with other biological components to maintain a biologically appropriate pH in an animal. The biological components are effective in maintaining a biologically appropriate pH even at hypothermic temperatures and at essentially bloodless conditions. Examples of the normal biological components include, but are not limited to carboxylic acids, salt and ester thereof. Carboxylic acids have the general structural formula of RCOOX, where R is an alkyl, alkenyl, or aryl, branched or straight chained, containing 1 to 30 carbons which carbons may be substituted, and X is hydrogen or sodium or other biologically compatible ion substituent which can attach at the oxygen position, or is a short straight or branched chain alkyl containing 1-4 carbons, e.g., —CH₃, —CH₂ CH₃. Examples of carboxylic acids and carboxylic acid salts include, but are not limited to, lactate and sodium lactate, citrate and sodium citrate, gluconate and sodium gluconate, pyruvate and sodium pyruvate, succinate and sodium succinate, and acetate and sodium acetate.

### Coagulation Enhancers

In certain embodiments, the nanoparticle composition may further comprise one or more coagulation enhancers. Examples of coagulation factors include, but are not limited to, factor VII, thrombin, platelets and tranexemic acid. These factors may be from natural or non-natural sources. In certain embodiments, factor 7 is added to the nanoparticle composition at a concentration of 70-150 IU/kg, prothrombin complex is added to the nanoparticle composition at a concentration of 15-40 IU/kg, and fibrinogen is added to the nanoparticle composition at a concentration of 50-90 mg/kg. Naturally-derived or synthetic platelets or platelet substitutes may also be added.

### Antioxidants

In certain embodiments, the nanoparticle composition may further comprise one or more antioxidants. Examples of antioxidants include, but are not limited to, sodium hydrogen sulfite, sodium sulfite, sodium pyrosulfite (e.g., sodium metabisulfite), rongalite (CH₂OHSO₂Na), ascorbic acid, sodium ascorbate, erythorbic acid, sodium erythorbate, cysteine, cysteine hydrochloride, homocysteine, glutathione, thioglycerol, α-thioglycerin, sodium edetate, citric acid, isopropyl citrate, potassium dichloroisocyanurate, sodium thioglycolate, sodium pyrosulfite 1,3-butylene glycol, disodium calcium ethylenediaminetetraacetate, disodium ethylenediaminetetraacetate, an amino acid sulfite (e.g, L-lysine sulfite), butylhydroxyanisole (BHA), butylhydroxytoluene (BHT), propyl gallate, ascorbyl palmitate, vitamin E and derivatives thereof (e.g., dl-α-tocopherol, tocopherol acetate, natural vitamin E, d-δ-tocopherol, mixed tocopherol, and trolox), guaiac, nordihydroguaiaretic acid (NDGA), L-ascorbate stearate esters, soybean lecithin, palmitic acid ascorbic acid, benzotriazol, and pentaerythrityl-tetrakis [3-(3,5-di-t-butyl-4-hydroxyphenyl) propionate]2-mercaptobenzimidazole. Among those, preferable are sodium hydrogen sulfite, sodium sulfite, ascorbic acid, homocysteine, dl-α-tocopherol, tocopherol acetate, glutathione, and trolox.

### Other Components

In addition to the components discussed above, the nanoparticle composition may further comprise other additives that include, but are not limited to, antibiotics, such as penicillin, cloxacillin, dicloxacillin, cephalosporin, erythromycin, amoxicillin-clavulanate, ampicillin, tetracycline, trimethoprim-sulfamethoxazole, chloramphenicol, ciprofloxacin, aminoglycoside (e.g., tobramycin and gentamicin), streptomycin, sulfa drugs, kanamycin, neomycin, land monobactams; anti-viral agents, such as amantadine hydrochloride, rimantadin, acyclovir, famciclovir, foscarnet, ganciclovir sodium, idoxuridine, ribavirin, sorivudine, trifluridine, valacyclovir, valgancyclovir, pencyclovir, vidarabin, didanosine, stavudine, zalcitabine, zidovudine, interferon alpha, and edoxudine; anti-fungal agents such as terbinafine hydrochloride, nystatin, amphotericin B, griseofulvin, ketoconazole, miconazole nitrate, flucytosine, fluconazole, itraconazole, clotrimazole, benzoic acid, salicylic acid, voriconazole, caspofungin, and selenium sulfide; vitamins, amino acids, vessel expanders such as alcohols and polyalcohols, surfactants, antibodies against harmful cytokines such as tumor necrosis factor (TNF) or interleukins, and mediators of vascular potency and immunomoduators, such as prostaglandins, leukotrienes, pro-opiomelanocortin fragments and platelet activating factors.

In certain embodiments, the nanoparticle composition may further contain beneficial anions such as lactate or glutamate. Hypertonic lactate containing compositions have been found to be effective in reducing brain edema in patients with acute hemodynamic distress. In one embodiment, the nanoparticle composition contains 250 to 2400 Mm of lactic acid or lactate. In another embodiment, the nanoparticle composition contains 250 to 2400 Mm of lactic acid or lactate and 2 to 10 Mm potassium.

In certain other embodiments, the nanoparticle composition may contain substituted cations. For example, the nanoparticle composition may contain choline to substitute sodium ions.

In some other embodiments, the nanoparticle composition further comprises a potassium channel blocker, which is capable of inhibiting programmed cell death by preventing potassium efflux.

In certain embodiments, the nanoparticle composition further contains anti-cancer drugs and/or intracellular signal molecules, such as Camp and diacylglycerol. In other embodiments, the nanoparticle composition further contain one or more organelles or organelle components such as endoplasmic reticulum, ribosomes, and mitochondria in whole or in part.

In other embodiments, the nanoparticle composition may be combined with red blood cells, modified red blood cells or other cellular components of blood.

In yet other embodiments, the nanoparticle composition further comprises proopiomelanocortin fragments, such as β-endorphin and melanocyte stimulating hormone, enkephalins or opiates to modify the immune response and to provide analgesia. β-endorphin may also be used at a final concentration of 0.01-100 nm, preferably 0.1-10 nm, more preferably about 1 nm, to modulate neutrophilic function in the septic state.

In yet other embodiments, the nanoparticle composition further comprises one or more neurotropic agents for treatment of psychiatric disease or prevention of psychiatric disease.

### IV. Preparation of the Nanoparticle Composition

The nanoparticle composition may be prepared by mixing the lipophilic or hydrophobic component, the emulsifier, the aqueous carrier, and any other components to form an emulsion. Commonly used mixing methods include, but are not limited to, stirring, shaking, homogenization, vibration, microfluidization and sonication.

An exemplary homogenizer is the APV2000 homogenizer (SPX Corporation). Emulsions may be formed at a pressure setting of ~15,000 to 20,000 psi for nanoemulsions <100 nm or ~22,000 to 28,000 psi for larger micelle emulsions of ~300 nm. Multiple rounds (cycles) of homogenization may be needed to produce micelles of the desired sizes. The number of homogenization cycles may vary depending upon the formulation and may require, for example, 6-cycles, 8 cycles, 10 cycles, 12 cycles, 15 cycles.

A suitable particle analyzer and/or zeta potential analyzers may be used to evaluate and monitor the size and stability of the micelle compositions. Exemplary analyzers include the Malvern Zetasizer Nano ZS, which can provide both size and zeta potential measurements

In one embodiment, the nanoparticle composition is formed by mixing a pre-formed lipid emulsion from the above described components with the aqueous carrier. In addition, the nanoparticle composition can be carried in erythrocyte ghosts. Specifically, the emulsion should be prepared in manners that allow the lipophilic gases dissolving into the lipophilic or hydrophobic portion of the emulsion but not forming microbubbles which may increase the risk of gas embolization.

In certain embodiments, albumin or albumin polymers or albumin polymers conjugated with amino acids or peptides is added to the nanoparticle composition in an amount of 2-40% (w/v), about 2-20% (w/v), about 4-10% (w/v), or about 5% (w/v). The albumin or albumin polymers or albumin polymers conjugated with amino acids or peptides is added to the nanoparticle composition after the formation of micelles. In one embodiment, the lipophilic or hydrophobic component, the emulsifier, the aqueous carrier and any other non-albumin components are mixed to form an emulsion. Albumin, albumin polymers or albumin polymers conjugated with amino acids or peptides is then dissolved in the emulsion at the desired concentration.

In some embodiments, Part A or the mixture of Part A and Part B, is loaded with oxygen, nitric oxide, carbon monoxide, xenon, argon, hydrogen sulfide other hydrophobic gases or mixtures of these gases prior to use. These gases may be used to deliver oxygen for aerobic metabolism after the initial bolus, provide an initial carbon monoxide bolus to protect against MODS, to open vessels in vascular diseases or states involving vascular constriction or obstruction, xenon or argon to protect against the effects of traumatic brain injury or seizures, or hydrogen sulfide to promote long-term tissue preservation. Nitric oxide loaded micelles may also be used as an anti-hypertensive medication. Either Part A, Part B, or the mixture of Part A and Part B can be sterilized by autoclaving.

In some embodiments, the soybean oil, which enhances clotting, is replaced with chia bean oil which is anti-inflammatory and reduces clotting. In one embodiment, a nanoparticle composition with soybean oil is used in initial phase of the infusion in which bleeding is occurring. A nanoparticle composition with chia bean oil is used for later stages of the infusion when bleeding is no longer an issue an issue.

In some other embodiments, the glycerol in Part A is replaced with mannitol. In other embodiments, the egg phospholipids are replaced with α-phosphatidylcholine to eliminate a potential source of protein contamination and anaphylaxis (due to contamination of egg phospholipid with egg protein). In yet other embodiments, the amino acids in Part B of Recipe 2 are replaced with N-acetyl amino acids. In one embodiment, the nanopartic nanoparticle composition is a non-oxygenated nanoparticle composition. As used herein, the term “non-oxygenated nanoparticle composition” refers to a formulation that is prepared in atmospheric air and is not loaded with oxygen by any oxygenation device or method.

In some embodiments, the nanoparticle composition comprises); an amphiphilic emulsifier in an amount of 6%-18% (w/v); a lipophilic or hydrophobic component in an amount of 15-35% (w/v), a polar liquid carrier; and one or more electrolytes, wherein the amphiphilic emulsifier forms lipid carrying micelles (LMs) having a lipophilic or hydrophobic core comprising the lipophilic or hydrophobic component in the polar liquid carrier, and wherein the LMs have diameters in the range of 20-140 nm. In some further embodiments, the nanoparticle composition comprises LMs with diameters in the range of 30-140 nm, 30-130 nm, 30-120 nm, 30-100 nm, 30-90 nm, 30-80 nm, 30-70 nm, 40-140 nm, 40-130 nm, 40-120 nm, 40-100 nm, 40-90 nm, 40-80 nm, 50-140 nm, 50-130 nm, 50-120 nm, 50-100 nm, 50-90 nm, 50-80 nm, 50-70 nm, 60-140 nm, 60-130 nm, 60-120 nm, 60-100 nm, 60-90 nm, 60-80 nm, 80-140 nm, 80-130 nm, 80-120 nm, 80-110 nm, 80-100 nm, 100-140 nm, 100-130 nm, 100-120 nm, 100-110 nm, 120-140 nm, 120-130 nm or 130-140 nm, as measured by electron microscopy method.

In some further embodiments, the nanoparticle composition further comprises liposomes with diameters of in the range of 1-30 nm, 1-25 nm, 1-20 nm, 1-15 nm, 1-10 nm, 3-30 nm, 3-25 nm, 3-20 nm, 3-15 nm, 3-10 nm, 5-30 nm, 5-25 nm, 5-20 nm, 5-15 nm, 5-10 nm, 7-30 nm, 7-25 nm, 7-20 nm, 7-15 nm, 7-10 nm, 10-30 nm, 10-25 nm, 10-20 nm, 10-15 nm, 15-30 nm, 15-25 nm, 15-20 nm, 20-30 nm, 20-25 nm or 25-30 nm, as measured by electron microscopy.

In some further embodiments, the nanoparticle composition comprises nanoparticles (including micelles and liposomes) with an average diameter in the range of about 70-160 nm, 70-150 nm, 70-140 nm, 70-130 nm, 70-120 nm, 70-100 nm, 70-90 nm, 80-160 nm, 80-150 nm, 80-140 nm, 80-130 nm, 80-120 nm, 80-100 nm, 80-90 nm, 90-160 nm, 90-150 nm, 90-140 nm, 90-130 nm, 90-120 nm, 90-100 nm, 90-98 nm, 92-96 nm, or 95-100 nm, as measure by dynamic light scattering.

In some embodiments, the nanoparticle composition comprises a mixture of LMs with diameters in the range of 30-500 nm and liposomes with diameters in the range of 1-30 nm, as measured by electron microscopy.

In some embodiments, the nanoparticle composition comprises a mixture of LMs and liposomes, wherein the average diameter of all particles is in the range of 5-25 nm, 5-20 nm, 5-15 nm, 5-10 nm, 10-25 nm, 10-20 nm, 10-15 nm, 15-25 nm, 15-20 nm or 20-25 nm, as determined by electron microscopy.

In some embodiments, the nanoparticle composition comprises about 12% egg lecithin and comprises LMs with diameters of about 30-500 nm and liposomes with diameters of about 1-25 nm, as determined by electron microscopy. In some embodiments, the nanoparticle composition comprises about 12% egg lecithin and comprises LMs with diameters of about 40-100 nm and liposomes with diameters of about 7-20 nm, as determined by electron microscopy.

In some embodiments, the nanoparticle composition comprises about 5-35% soybean oil and 0.5-20% egg lecithin and comprises LMs with diameters of about 15-800 nm and liposomes with diameters of about 1-300 nm, as determined by electron microscopy.

In some embodiments, the nanoparticle composition comprises about 5-25% soybean oil and 0.5-1.5% egg lecithin and comprises LMs with diameters of about 30-400 nm and liposomes with diameters of about 1-150 nm, as determined by electron microscopy.

In some embodiments, the nanoparticle composition comprises about 5-25% soybean oil and 0.5-1.5% egg lecithin and comprises LMs with diameters of about 30-400 nm and liposomes with diameters of about 1-150 nm, as determined by electron microscopy.

In some embodiments, the nanoparticle composition comprises about 12% egg lecithin and comprises LMs with diameters of about 40-100 nm and liposomes with diameters of about 7-20 nm, as determined by electron microscopy.

In some embodiments, the nanoparticle compositions described above further comprise glycerin in the amount of 1-10%, 1-8%, 1-5%, 1-3%, 1-2%, 5-10%, 2-8%, 2-5%, 2-3%, 3-10%, 3-8%, 3-5%, 5-10%, 5-8% or 8-10% (w/v)/

In some embodiments, the nanoparticle composition described above further comprises NaCl at a final concentration of 50-200 mM, 50-150 mM, or 50-100 mM.

In some embodiments, the nanoparticle composition is prepared under atmospheric air without enrichment of oxygen, carbon monoxide, nitric oxide or xenon.

In certain embodiments, the nanoparticle composition may be loaded with a lipophilic gas prior to clinical application. Examples of such gases include, but are not limited to, oxygen, xenon, argon, nitric oxide, carbon monoxide, hydrogen sulfide. The gases are present in amounts sufficient for regulation of vascular function and cellular em nanoparticle bolism. As used herein, “a nanoparticle composition loaded with a lipophilic gas” refers to a composition that has been subjected to a process to increase the content of such lipophilic gas in the nanoparticle composition. A nanoparticle composition may be loaded with a lipophilic gas by bubbling the lipophilic gas through the nanoparticle composition for a desired period of time, or by agitating the nanoparticle composition in the presence of the lipophilic gas under pressure.

In one embodiment, the nanoparticle composition is oxygenated by bubbling pure oxygen or a gas with an oxygen content in the range of 21% to 100% (v/v), preferably 40% to 100% (v/v), more preferably 60% to 100% (v/v), and most preferably 80% to 100% (v/v), through the mixture for a period of 30 seconds or longer, preferably 1-15 minutes, more preferably 1-5 minutes. Oxygen may also be added under pressure followed by a reduction of the pressure to one atmosphere. In one embodiment, the nanoparticle composition is oxygenated immediately prior to application. The nanoparticle composition may be oxygenated using portable oxygen tanks or portable oxygen concentrators, such the Evergo Portable Pulse Dose Oxygen concentrator produced by Philips Healthcare at Andover, MA.

Another method could be allowing the emulsion to equilibrate with an atmosphere filled with the gas that is to be added. In most cases a bubble trap would be necessary to remove bubbles that could become gas emboli. The equilibration time for a nanoparticle composition of a particular composition may be determined experimentally.

In some embodiments, the nanoparticle composition comprises an oxygenated lipid emulsion. As used herein, the term “oxygenated lipid emulsion” or “oxygenated nanoparticle composition” refers to a specific type of gassed lipid emulsion or gassed fluid which has been forced to absorb oxygen such that the total concentration of oxygen contained therein is greater than that present in the same liquid at atmospheric equilibrium conditions.

### EXAMPLES

The following example is put forth so as to provide those of ordinary skill in the art with a complete disclosure and description of how to carry out the method of the present application and is not intended to limit the scope of the invention. Efforts have been made to ensure accuracy with respect to numbers used (e.g., amounts, temperature, etc.), but some experimental error and deviation should be accounted for. Unless indicated otherwise, parts are parts by weight, molecular weight is weight average molecular weight, temperature is in degrees Centigrade, and pressure is at or near atmospheric.

### Example 1: Preparation of Nanoparticle Composition No. 1

### Preparation of Part A

soybean oil 20%=20 grams/100 ml
egg yolk phospholipids 1.2 grams/100 ml
sonicate or microfluidize and/or homogenize to produce a nanoparticle composition.

### Preparation of Part B

Glycerol 2.25%
NaCl 77 mM

### Example 2: Preparation of Nanoparticle Composition No. 2

### Preparation of Part A

soybean oil 10%-20%=10-20 grams/100 ml.
egg yolk phospholipids 0.6% to 12%=0.6 to 12 grams
sonicate or microfluidize and/or homogenize to produce a nanoparticle composition.

### Preparation of Part B

Glycerol 2.25%
NaCl 77 mM

### Example 3: Preparation of Nanoparticle Composition No. 3

### Preparation of Part A

soybean oil 10%-20%=10-20 grams
egg yolk phospholipids 0.6% to 12%=0.6 to 12 grams
sonicate or microfluidizer and/or homogenize to produce a nanoparticle composition.

### Preparation of Part B

Glycerol 2.25%
NaCl 77 mM
L-Histidine 1 mM

### Example 4: Preparation of Nanoparticle Composition No. 4

### Preparation of Part A

egg yolk phospholipids 0.6% to 12%=0.6 to 12 grams
sonicate or microfluidize and/or homogenize to produce a nanoparticle composition.

### Preparation of Part B

Glycerol 2.25%
NaCl 77 mM

### Example 5: Preparation of Nanoparticle Composition No. 5

### Preparation of Part A

egg yolk phospholipids 0.6% to 12%=0.6 to 12 grams
sonicate or microfluidize and/or homogenize to produce a nanoparticle composition.

### Preparation of Part B

Glycerol 2.25%
NaCl 77 mM
L-Histidine 1 mM

### Example 6: Preparation of Nanoparticle Composition No. 6

### Preparation of Part A

soybean oil 10%-20%=10-20 grams
egg yolk phospholipids 0.6% to 12%=0.6 to 12 grams
sonicate or microfluidize and/or homogenize to produce a nanoparticle composition.

### Preparation of Part B

Glycerol 2.25%
NaCl 77 mM
L-Histidine 77 mM
Albumin 5% (w/v)

### Example 7: Preparation of Nanoparticle Composition No. 7

### Preparation of Part A

soybean oil 10%-20%=10-20 grams
egg yolk phospholipids 0.6% to 12%=0.6 to 12 grams
sonicate or microfluidize and/or homogenize to produce a nanoparticle composition.

### Preparation of Part B

NaCl 103 mM
Na-L-lactate 34 mM
L-Histidine 1 mM

Part A may be used alone, or mixed with Part B within 24 hours of use, or premixed. Either or both parts may also be lyophilized and water can be added at the time of use. In some embodiments, glycerin was added to the final product at a final concentration of 1.13% (w/v) or 2.25% (w/v).

### Example 7: Treatment of Hyperprocalcitonemia with the Nanoparticle Composition of the Present Application

Septic patients with hyperprocalcitonemia were treated with the nanoparticle composition No. 1 described in Example 1. The nanoparticle composition treatment consistently reduced the concentration of procalcitonin in the bloodstream and improved the function of multiple organs. The blood procalcitonin concentration was measured prior to the nanoparticle composition treatment followed by subsequent measurements over the next 48 hours. The results are shown below in Table 1.

TABLE 1
Procalcitonin levels in patients treated with the nanoparticle
composition of the present application

| | Amount of | | |
|---|---|---|---|
| | nanoparticle | Procalcitonin | Procalcitonin |
| | composition | Before | After |
| Patient ID | given (ml) | treatment | treatment |
| 01-012-002 | 870 | 45.62 | 13 |
| 01-012-004 | 1300 | 423.1 | 130.11 |
| 01-012-005 | 192 | 50.06 | 44.5 |
| 01-012-006 | 300 | 44.75 | 22.35 |
| 01-012-007 | 341 | 47.6 | 28.53 |
| 01-012-008 | 1118.1 | 22.02 | 12.27 |
| 01-012-009 | 800 | 5.94 | 0.93 |
| 01-006-002 | 300 | 10.63 | 4.36 |
| 01-006-003 | 1459 | 12.41 | 7.97 |
| 01-006-004 | 416 | 70.49 | 14.6 |
| 01-006-005 | 673 | 639.12 | 293.97 |
| 01-004-002 | 560 | 19.17 | 6.51 |
| 02-011-003 | 350 | 122 | 20.03 |
| Mean ± SE | 632.24 ± 123.02 | 116.38 ± 53.19 | 46.09 ± 22.66 * |

* p < 0.0001 by two-tailed Student's t test on arcsin transformed data

These data show that there was a highly significant reduction of procalcitonin within 48 hours after infusion of the nanoparticle composition of VBI-S. This reduction was not simply due to improvement of the patients because patients 01-012-002, 01-012-005, and 01-006-003 did not survive the 48-hour treatment period yet still had a decrease in procalcitonin. The reduction was not due to dilution. Because the sodium and blood urea nitrogen concentrations did not change and the average volume of the nanoparticle composition given was only 632 ml.

This reduction of procalcitonin by the nanoparticle composition of the present application was unexpected because infusion of liposome composition has been shown to increase the blood concentration of inflammatory mediators, such as tumor necrosis factor, and interleukin 6, which have been known to increase procalcitonin. Therefore, one skilled in the art would expect that the infusion of the nanoparticle composition of the present application would lead to an increase in procalcitonin, yet we observed a definitive and consistent reduction.

The terms and descriptions used herein are set forth by way of illustration only and are not meant as limitations. Those skilled in the art will recognize that many variations are possible within the spirit and scope of the invention as defined in the following claims, and their equivalents, in which all terms are to be understood in their broadest possible sense unless otherwise indicated.

What is claimed is:

1. A method for treating hyperprocalcitoninemia in a subject, comprising:
administering to a subject in need of treatment, an effective amount of nanoparticle composition comprising:
an amphiphilic emulsifier in an amount of 0.1%-100% (w/v);
a lipophilic or hydrophobic component in an amount of 0-35% (w/v);
a polar liquid carrier; and
one or more electrolytes,
wherein the nanoparticle composition comprises liposomes and/or micelles having diameters in the range of 1-800 nm.

2. The method of claim 1, wherein the lipophilic or hydrophobic component is selected from the group consisting of soybean oil, chia bean oil and algae oil.

3. The method of claim 1, wherein the amphiphilic emulsifier is selected from the group consisting of phospholipids, α-phosphatidylcholine, amphiphilic peptides and amphiphilic polymers.

4. The method of claim 1, wherein the amphiphilic emulsifier is selected from the group consisting of egg yolk lecithin and soybean lecithin.

5. The method of claim 1, wherein the polar liquid carrier is selected from the group consisting of water, a water-based solution and a non-aqueous polar liquid.

6. The method of claim 1, wherein the non-aqueous polar liquid is selected from the group consisting of dimethyl sulfoxide, polyethylene glycol and polar silicone liquids.

7. The method of claim 1, wherein the electrolytes are selected from the group consisting of one or more of sodium chloride, sodium hydrogen carbonate, sodium citrate, sodium lactate, sodium sulfate, sodium dihydrogen phosphate, disodium hydrogen phosphate, sodium acetate, sodium glycerophosphate, sodium carbonate, an amino acid sodium salt, sodium propionate, sodium hydroxybutyrate, sodium gluconate, potassium chloride, potassium acetate, potassium gluconate, potassium hydrogen carbonate, potassium glycerophosphate, potassium sulfate, potassium lactate, potassium iodide, potassium dihydrogen phosphate, dipotassium hydrogen phosphate, potassium citrate, an amino acid potassium salt, potassium propionate, potassium hydroxybutyrate, calcium chloride, calcium gluconate, calcium lactate, calcium glycerophosphate, calcium pantothenate, calcium acetate, magnesium chloride, magnesium sulfate, magnesium glycerophosphate, magnesium acetate, magnesium lactate, an amino acid magnesium salt, ammonium chloride, zinc sulfate, zinc chloride, zinc gluconate, zinc lactate, zinc acetate, iron sulfate, iron chloride, iron gluconate, copper sulfate, and manganese sulfate.

8. The method of claim 1, wherein the nanoparticle composition is administered either intravenously, intra-arterially, intraosseously or intracardially.

9. The method of claim 1, wherein the nanoparticle composition is an oxygenated nanoparticle composition with an oxygen content of 1-50,000 ml O₂/100 ml nanoparticle composition.

10. The method of claim 1, wherein the nanoparticle composition has an emulsifier: lipophilic or hydrophobic component ratio (w/w) between about 1:200 to about 1:1.7.

11. The method of claim 1, wherein the nanoparticle composition comprises micelles and liposomes, wherein the micelles in the nanoparticle composition have diameters in the range of 15-200 nm as measured by electron microscopy and wherein the liposomes in the nanoparticle composition have diameters in the range of 1-25 nm as measured by electron microscopy.

12. The method of claim 1, wherein the nanoparticle composition has a concentration of magnesium ion in a subphysiological range.

13. The method of claim 1, wherein the nanoparticle composition further comprises one or more selected from the group consisting of a crystalloid agent, an oncotic agent, an anti-inflammatory agent, an immunomodulatory agent, and a lipophilic gas.

14. The method of claim 1, wherein the nanoparticle composition further comprises glycerin.

15. A method for treating a disease or condition associated with elevated procalcitonin a in a subject, comprising:
administering to a subject in need of treatment, an effective amount of nanoparticle composition comprising:
an amphiphilic emulsifier in an amount of 0.1%-100% (w/v);
a lipophilic or hydrophobic component in an amount of 0-35% (w/v);
a polar liquid carrier; and
one or more electrolytes,
wherein the nanoparticle composition comprises liposomes and/or micelles having diameters in the range of 1-800 nm.

16. The method of claim 15, wherein the disease or condition associated with elevated procalcitonin is selected from the group consisting of: chronic kidney failure, acute kidney failure, chronic liver failure, acute liver failure, blood loss, sepsis, cardiac failure, injury to the neurological system, status epilepticus, chronic respiratory failure, acute respiratory failure, sarcopenia of aging, dementia, orthopedic trauma, non-orthopedic trauma, surgical procedures, myocardial infarction, autism, ischemic stroke, Parkinson's disease, vasculitis, bone fractures, depression and cancers.

17-31. (canceled)

32. A method for treating aging, comprising:
administering to a subject in need of treatment, an effective amount of nanoparticle composition comprising:
an amphiphilic emulsifier in an amount of 0.1%-100% (w/v);
a lipophilic or hydrophobic component in an amount of 0-35% (w/v);
a polar liquid carrier; and
one or more electrolytes,
wherein the nanoparticle composition comprises liposomes and/or micelles having diameters in the range of 1-800 nm.
